(19) United States
(12) Patent Application Publication
(10) Pub. No.: US 2020/0172927 A1
(43) Pub. Date: Jun. 4, 2020

(54) REPROGRAMMING METABOLISM BY INHIBITING VHL FOR TREATMENT OF NEURODEGENERATION

(71) Applicant: The Trustees of Columbia University in the City of New York, New York, NY (US)

(72) Inventors: Stephen H. TSANG, New York, NY (US); Xuan CUI, New York, NY (US); Pei-Yin SU, New York, NY (US); Sally JUSTUS, New York, NY (US)

(21) Appl. No.: 16/613,616

(22) PCT Filed: May 15, 2018
(86) PCT No.: PCT/US18/32717

§ 371 Date: Nov. 14, 2019

Related U.S. Application Data
(60) Provisional application No. 62/506,207, filed on May 15, 2017.

(51) Int. Cl. C12N 15/86; C12N 9/22; C07K 14/47; A61K 48/00; A61K 9/00; A01K 67/027; A61P 23/00

(52) U.S. Cl. CPC C12N 15/86; C12N 9/22; C07K 14/4703; A61K 48/005; A61K 9/0019; A01K 2267/0306; A61P 23/00; A01K 2217/15; A01K 2217/203; A01K 2217/206; A01K 2227/105; A01K 67/0275

(57) ABSTRACT

The present disclosure relates to methods and compounds for promoting anabolic pathways in neuronal cells leading to improved neuronal survival. In particular, the present disclosure relates to inhibiting YHL/Vhl to promote glycolysis and neuronal survival in a variety of neurodegenerative conditions, and specifically in retinitis pigmentosa.

### CROSS-REFERENCE TO RELATED APPLICATION

The present application claims priority to U.S. Provisional Patent Application No. 62/506,207 filed on May 15, 2017, which is incorporated herein by reference in its entirety.

### FIELD OF THE INVENTION

The present disclosure relates to methods and compounds for promoting anabolic pathways in neuronal cells leading to improved neuronal survival. In particular, the present disclosure relates to inhibiting VHL/Vhl to promote glycolysis and neuronal survival in a variety of neurodegenerative conditions, and specifically in retinitis pigmentosa.

### BACKGROUND

Retinitis pigmentosa (RP) is an incurable neurodegenerative condition that leads to progressive photoreceptor dysfunction, dysmorphosis and symptoms such as nyctalopia, tunnel vision and eventually, blindness. This disease is estimated to affect nearly 1 million people worldwide and leads to a substantial decrease in the ability of affected individuals to lead independent lives and conduct activities of daily living. A heterogeneous genetic condition, RP is linked to more than 60 genes, most of which are exclusively expressed in rod photoreceptors. Daiger, et al., (2008) Mutations in known genes account for 58% of autosomal dominant retinitis pigmentosa (adRP). Adv. Exp. Med. Biol., 613, 203-209. Due to the genetic diversity of RP, any therapy that is gene specific can only benefit a small fraction of patients with RP. There is currently no effective therapeutic option for patients with RP or any other patient with a retinal degenerative disease, including atrophic age-related macular degeneration (AMD), which affects more than 1.5 million individuals in the United States (8).

Thus, there is an urgent need for additional therapeutics as well as more broadly effective gene therapies for alleviating retinal degenerative diseases such as RP and AMD, and more broadly for promoting neuronal survival in neurodegenerative diseases such as glaucoma, Alzheimer's, Parkinson's, Huntington's, Amyotrophic lateral sclerosis (ALS), Lewy body dementia, and similar neurodegenerative conditions or other conditions that would benefit from upregulating anabolism and downregulating catabolism to promote neuronal survival.

### SUMMARY

In certain embodiments, the present disclosure provides a method of increasing glycolysis in a neuronal cell comprising inhibiting or decreasing level and/or activity of VHL in the neuronal cell.

In certain embodiments, the present disclosure provides a method of increasing neuronal survival in patient(s) in need thereof, comprising altering glycolysis by inhibiting or decreasing level and/or activity of VHL in the neuronal cell.

In certain embodiments, the present disclosure provides a method of increasing photoreceptor survival comprising altering glycolysis by inhibiting or decreasing level and/or activity of VHL in a photoreceptor cell.

In certain embodiments, the neuronal cell is a cone cell or a rod cell, or a combination of cone cells, rod cells, and/or other retinal cells.

In additional embodiments, the method comprises administering an effective amount of an inhibitor of VHL.

The inhibitor of VHL may be a small molecule inhibitor, such as VH298 ((2S,4R)-1-((S)-2-(1-cyanocyclopropanecarboxamido)-3,3-dimethylbutanoyl)-4-hydroxy-N-(4-(4-methylthiazol-5-yl)benzyl)pyrrolidine-2-carboxamide), VH032 ((2S,4R)-1-((S)-2-acetamido-3,3-dimethylbutanoyl)-4-hydroxy-N-(4-(4-methylthiazol-5-yl)benzyl)pyrrolidine-2-carboxamide), VH125, or combinations thereof.

The inhibitor of VHL may be proteins, nucleic acids, chemicals and combinations thereof. In additional embodiments, the nucleic acid is selected from the group consisting of antisense oligonucleotide, siRNA, shRNA, gRNA and combinations thereof.

In additional embodiments, the decreasing comprises administering an effective amount of any combination of inhibitors of VHL.

In additional embodiments, the patient is suffering from one or more retinal degenerative diseases such as retinitis pigmentosa (RP), age-related macular degeneration (AMD), or glaucoma, or one or more neurodegenerative diseases including Alzheimer's, Parkinson's, Huntington's, Amyotrophic lateral sclerosis (ALS), or Lewy body dementia.

In certain embodiments, the present disclosure provides a method of increasing photoreceptor survival in a patient in need thereof, comprising administering to the subject a therapeutically effective amount of:
a recombinant adeno-associated viral (AAV) vector encoding an inhibitor of Vhl, or other metabolic reprogramming agent, or an inhibitor or activator of anabolism.

In certain embodiments, the recombinant AAV vector is an AAV2 vector. In additional embodiments, the AAV vector is an AAV8 vector. In certain embodiments, the AAV vectors are administered by intravitreal injection. In additional embodiments, the AAV vectors are administered by subretinal injection.

In certain embodiments, the present disclosure provides a method of increasing photoreceptor survival in a patient in need thereof, comprising administering to the patient a therapeutically effective amount of:
(a) a first recombinant adeno-associated viral (AAV) vector, wherein the first recombinant AAV comprises, (i) a first sequence(s) encoding at least one guide RNA that hybridizes to the endogenous Vhl gene in the patient, and,
(b) a second recombinant AAV viral vector comprising a nucleic acid sequence encoding a Cas nuclease; wherein the Cas nuclease cleaves the endogenous Vhl gene creating a Vhl knockout of the endogenous Vhl gene in the patient.

In certain embodiments, the recombinant AAV vector is an AAV2 vector. In additional embodiments, the AAV vector is an AAV8 vector. In certain embodiments, the Cas nuclease is Cas9. In certain embodiments, the AAV vectors are administered by intravitreal injection. In additional embodiments, the AAV vectors are administered by subretinal injection.

In additional embodiments, the present disclosure provides a method of increasing neuronal survival in a patient in need thereof, comprising administering to the patient a therapeutically effective amount of:
a recombinant adeno-associated viral (AAV) vector encoding an inhibitor of Vhl, or other metabolic reprogramming agent, or an inhibitor or activator of anabolism, to at least one neuron in the patient.

In certain embodiments, the recombinant AAV vector is an AAV2 vector. In additional embodiments, the AAV vector is an AAV8 vector. In additional embodiments, the AAV vectors are administered by intravitreal injection. In additional embodiments, the AAV vectors are administered by subretinal injection.

In additional embodiments, the present disclosure provides a method of increasing neuronal survival in a patient in need thereof, comprising administering to the patient a therapeutically effective amount of:
(a) a first recombinant adeno-associated viral (AAV) vector, wherein the first recombinant AAV comprises: (i) a first sequence(s) encoding at least one guide RNA that hybridizes to the endogenous Vhl gene in the patient, and,
(b) a second recombinant AAV viral vector comprising a nucleic acid sequence encoding a Cas nuclease; wherein the Cas nuclease cleaves the endogenous Vhl gene creating a Vhl knockout of the endogenous Vhl gene in the patient.

In certain embodiments, the recombinant AAV vector is an AAV2 vector. In additional embodiments, the AAV vector is an AAV8 vector. In additional embodiments, the Cas nuclease is Cas9. In additional embodiments, the AAV vectors are administered by intravitreal injection. In additional embodiments, the AAV vectors are administered by subretinal injection.

In additional embodiments, the present disclosure provides a method of increasing glycolysis in a neuronal cell in patient(s) in need thereof, comprising administering a therapeutically effective amount of:
a recombinant adeno-associated viral (AAV) vector encoding an inhibitor of Vhl, or other metabolic reprogramming agent, or an inhibitor or activator of anabolism, to at least one neuronal cell in the patient.

In certain embodiments, the recombinant AAV vector is an AAV2 vector. In additional embodiments, the AAV vector is an AAV8 vector. In additional embodiments, the AAV vectors are administered by intravitreal injection. In additional embodiments, the AAV vectors are administered by subretinal injection.

In additional embodiments, the present disclosure provides a method of increasing glycolysis in a neuronal cell in a patient in need thereof, comprising administering to the patient a therapeutically effective amount of:
(a) a first recombinant adeno-associated viral (AAV) vector, wherein the first recombinant AAV comprises, (i) a first sequence(s) encoding at least one guide RNA that hybridizes to the endogenous Vhl gene in the patient and,
(b) a second recombinant AAV viral vector comprising a nucleic acid sequence encoding a Cas nuclease; wherein the Cas nuclease cleaves the endogenous Vhl gene creating a Vhl knockout of the endogenous Vhl gene in the patient's neuronal cell.

In certain embodiments, the recombinant AAV vector is an AAV2 vector. In additional embodiments, the AAV vector is an AAV8 vector. In additional embodiments, the Cas nuclease is Cas9. In additional embodiments, the AAV vectors are administered by intravitreal injection. In additional embodiments, the AAV vectors are administered by subretinal injection.

### BRIEF DESCRIPTION OF THE DRAWINGS

FIGS. 1A-1G are images and graphs showing comparisons of morphological and functional imaging modalities in control and RP patients.

FIG. 2 contains images that illustrate that photoreceptor cells are preserved after genome surgery.

FIGS. 3A-3B are graphs showing that photoreceptor function is preserved after genome surgery, as assessed by ERG. ERG in 4-, 6-, and 8-week old mice revealed higher scotopic, maximum, and photopic b-wave amplitudes (FIG. 3A) in the Vhl^{-/-} mice (white bar) compared with untreated mice (black bar), indicating that the function of the rods and cones is preserved in the treated group. Example raw data traces are also shown (FIG. 3B; curves with closed circles are for Vhl^{-/-} mice).

FIGS. 4A and 4B are a flow chart and graphs indicating that glycolysis is upregulated after genome surgery, resulting in higher levels of ATP. Mass spectrometry at 4 weeks revealed that the concentration of glucose, pyruvate, and FBP/GBP is higher in the Vhl^{-/-} group compared to the untreated mice, suggesting that glycolysis is upregulated after gene therapy. High-energy molecules like ATP and GTP were found in greater abundance in the Vhl^{-/-} group compared to the untreated mice.

FIG. 5 shows protein levels of HIF2a, and Glut1 in mice treated with VH298, an inhibitor of VHL. 6-week old B6 mice were orally fed for 3 days with VH298 in oil (10, 20, or 50 mg/kg) or oil only for the control group. Each group contained 3 mice. 4 hours after the last feeding, mice retinas were collected and processed immediately for immunoblot. Protein levels of HIF2a, and Glut1 were shown. B-actin was used as loading control.

### DETAILED DESCRIPTION

The present disclosure provides for compositions and methods for inhibiting VHL in the treatment or prophylaxis of retinal degenerative diseases, such as retinitis pigmentosa (RP), age-related macular degeneration (AMD), and glaucoma, or neurodegenerative diseases.

While more than 60 genes are implicated in RP, 36,000 cases are caused by mutations in the phosphodiesterase 6 (PDE6) gene. This gene encodes a rod-specific protein that regulates cyclic guanosine monophosphate (cGMP) levels and consequently plays a critical role in normal phototransduction. Mutations in PDE6 provoke a metabolic imbalance between catabolism and anabolism in rods that forces them to succumb to the consequences of elevated cGMP and Ca²⁺. Von Hippel-Lindau (VHL) protein is a transcriptional suppressor of glycolysis, a process associated with anabolic metabolism. Thus, we hypothesized that knocking out Vhl could enhance glycolysis and restore the balance between catabolic and anabolic metabolism, thus protecting against mutant PDE6-induced degeneration.

Methods and compositions of the present invention can be used for prophylaxis as well as treating a retinal degenerative disease or a neurodegenerative disease (e.g., amelioration of signs and/or symptoms of the retinal degenerative disease or neurodegenerative disease).

For prophylaxis, the present composition can be administered to a subject in order to prevent the onset of one or more symptoms of retinal degenerative disease. In one embodiment, the subject can be asymptomatic. A prophylactically effective amount of the agent or composition is administered to such a subject. A prophylactically effective amount is an amount which prevents the onset of one or more symptoms of the retinal degenerative disease.

The present compositions may be used in vitro or administered to a subject. The administration may be topical, intravenous, intranasal, or any other suitable route as described herein. The present compositions may be administered by intravitreal injection or subretinal injection.

The subject/patient treated with the present method and composition may suffer from one or more retinal degenerative diseases such as retinitis pigmentosa (RP), age-related macular degeneration (AMD), or glaucoma, or one or more neurodegenerative diseases including Alzheimer's, Parkinson's, Huntington's, Amyotrophic lateral sclerosis (ALS), or Lewy body dementia.

In accordance with the present invention, there may be numerous tools and techniques within the skill of the art, such as those commonly used in molecular immunology, cellular immunology, pharmacology, and microbiology. See, e.g., Sambrook et al. (2001) Molecular Cloning: A Laboratory Manual. 3rd ed. Cold Spring Harbor Laboratory Press: Cold Spring Harbor, N.Y.; Ausubel et al. eds. (2005) Current Protocols in Molecular Biology. John Wiley and Sons, Inc.: Hoboken, N.J.; Bonifacino et al. eds. (2005) Current Protocols in Cell Biology. John Wiley and Sons, Inc.: Hoboken, N.J.; Coligan et al. eds. (2005) Current Protocols in Immunology, John Wiley and Sons, Inc.: Hoboken, N.J.; Coico et al. eds. (2005) Current Protocols in Microbiology, John Wiley and Sons, Inc.: Hoboken, N.J.; Coligan et al. eds. (2005) Current Protocols in Protein Science, John Wiley and Sons, Inc.: Hoboken, N.J.; and Enna et al. eds. (2005) Current Protocols in Pharmacology, John Wiley and Sons, Inc.: Hoboken, N.J.

While not wishing to be bound by theory, aspects of the present invention relate to methods for increasing anabolism and decreasing catabolism in desired cells, in particular, in desired neuronal cells. Embodiments of the present invention relate to increasing glycolysis in neuronal cells, leading to improved neuronal cell survival. Additional embodiments of the present invention relate to methods of increasing photoreceptor cell survival in desired patient populations, including in patients with retinal degenerative diseases such as RP, AMD, and glaucoma. Indeed, we hypothesized that enhancing anabolic processes can confer beneficial effects on cells undergoing neurodegeneration, and we have validated this theory in a gene target, namely VHL. This strategy may also be used in combination with gene therapies and neurotrophic factor administration for heightened treatment efficacy.

By “neuronal” is meant to refer to and include any cells which compose the central or peripheral nervous system. (See, Dowling J E. The retina: an approachable part of the brain. Rev. ed. Cambridge, Mass.: Belknap Press of Harvard University Press; 2012.)

By “retinal” is meant to refer to and include any light-sensitive cells in the eye as well as the supporting cells that enable, facilitate, or are related to the phototransduction cascade.

By “nucleic acid” or “nucleic acid molecule” is meant to include a DNA, RNA, mRNA, cDNA, or recombinant DNA or RNA.

By “animal” is meant any member of the animal kingdom including vertebrates (e.g., frogs, salamanders, chickens, or horses) and invertebrates (e.g., worms, etc.). Preferred animals are mammals. Preferred mammalian animals include livestock animals (e.g., ungulates, such as bovines, buffalo, equines, ovines, porcines and caprines), as well as rodents (e.g., mice, hamsters, rats and guinea pigs), canines, felines and primates. By “non-human” is meant to include all animals, especially mammals and including primates other than human primates.

By “medium” or “media” is meant the nutrient solution in which cells and tissues are grown.

The term “pharmaceutically acceptable carrier”, as used herein means a pharmaceutically-acceptable material, composition or vehicle, such as a liquid or solid filler, diluent, excipient, solvent or encapsulating material, involved in carrying or transporting a chemical agent. The diluent or carrier ingredients should not be such as to diminish the therapeutic effects of the active compound(s).

The term “composition” as used herein means a product which results from the mixing or combining of more than one element or ingredient.

“Treating” or “treatment” of a state, disorder or condition includes:

(1) preventing or delaying the appearance of clinical symptoms of the state, disorder, or condition developing in a person who may be afflicted with or predisposed to the state, disorder or condition but does not yet experience or display clinical symptoms of the state, disorder or condition; or

(2) inhibiting the state, disorder or condition, i.e., arresting, reducing or delaying the development of the disease or a relapse thereof (in case of maintenance treatment) or at least one clinical symptom, sign, or test, thereof; or

(3) relieving the disease, i.e., causing regression of the state, disorder or condition or at least one of its clinical or sub-clinical symptoms or signs.

The benefit to a subject to be treated is either statistically significant or at least perceptible to the patient or to the physician.

“Treat” or “treating” may refer to administering a therapeutic agent, such as a composition containing any of the tissue-specific, e.g., neuronal or ocular targeted viral vectors, RNAi, shRNA or other Vhl inhibitors, combinations thereof, or similar compositions described herein, internally or externally to a subject or patient having one or more disease symptoms, or being suspected of having a disease or being at elevated at risk of acquiring a disease, for which the agent has therapeutic activity. Gene editing technology such as CRISPR/Cas9 methods may also be utilized to carry out tissue-specific reduction of Vhl or a combination thereof. Typically, the agent is administered in an amount effective to alleviate one or more disease symptoms in the treated subject or population, whether by inducing the regression of or inhibiting the progression of such symptom(s) by any clinically measurable degree. The amount of a therapeutic agent that is effective to alleviate any particular disease symptom (also referred to as the “therapeutically effective amount”) may vary according to factors such as the disease state, age, and weight of the patient, and the ability of the drug to elicit a desired response in the subject. Whether a disease symptom has been alleviated can be assessed by any clinical measurement typically used by physicians or other skilled healthcare providers to assess the severity or progression status of that symptom. While an embodiment of the present invention (e.g., a treatment method or article of manufacture) may not be effective in alleviating the target disease symptom(s) in every subject, it should alleviate the target disease symptom(s) in a statistically significant number of subjects as determined by any statistical test known in the art such as the Student's t-test, the chi^{t}-test, the U-test according to Mann and Whitney, the Kruskal-Wallis test (H-test), Jonckheere-Terpstra-test and the Wilcoxon-test.

“Treatment,” as it applies to a human, veterinary, or research subject, refers to therapeutic treatment, prophylactic or preventative measures, to research and diagnostic applications. “Treatment” as it applies to a human, veterinary, or research subject, or cell, tissue, or organ, encompasses transfection of any of the tissue-targeted viral vectors, delivery of RNAi, shRNA or other VHL inhibitors, combinations thereof, or similar compositions, including gene editing technology such as CRISPR/cas9 methods, which may be utilized to carry out tissue specific reduction of VHL, combinations thereof or related methods described herein as applied to a human or animal subject, a cell, tissue, physiological compartment, or physiological fluid.

A “therapeutically effective amount” means the amount of a compound that, when administered to an animal for treating a state, disorder or condition, is sufficient to effect such treatment. The “therapeutically effective amount” will vary depending on the compound, the disease and its severity and the age, weight, physical condition and responsiveness of the animal to be treated.

“Patient” or “subject” refers to mammals and includes human and veterinary subjects.

Acceptable excipients, diluents, and carriers for therapeutic use are well known in the pharmaceutical art, and are described, for example, in Remington: The Science and Practice of Pharmacy. Lippincott Williams & Wilkins (A. R. Gennaro edit. 2005). The choice of pharmaceutical excipient, diluent, and carrier can be selected with regard to the intended route of administration and standard pharmaceutical practice.

As used herein, the phrase “pharmaceutically acceptable” refers to molecular entities and compositions that are “generally regarded as safe”, e.g., that are physiologically tolerable and do not typically produce an allergic or similar untoward reaction, such as gastric upset, dizziness and the like, when administered to a human. Preferably, as used herein, the term “pharmaceutically acceptable” means approved by a regulatory agency of the Federal or a state government or listed in the U.S. Pharmacopoeia or other generally recognized pharmacopeias for use in animals, and more particularly in humans.

In certain embodiments, the methods of the present disclosure can be used for arresting progression of, or ameliorating, vision loss associated with photoreceptor degeneration including retinitis pigmentosa (RP) and age-related macular degeneration (AMD) in the subject. Vision loss linked to retinitis pigmentosa may include decrease in peripheral vision, central (reading) vision, night vision, day vision, loss of color perception, loss of contrast sensitivity, or reduction in visual acuity. The methods of the present disclosure can also be used to prevent, or arrest photoreceptor function loss, or increase photoreceptor function in the subject.

RP is diagnosed in part, through an examination of the retina and genetic testing. The eye exam usually reveals abnormal, intraretinal pigment migration. Additional tests for diagnosing RP include electroretinogram (ERG) and visual field testing.

Methods for measuring or assessing visual function, retinal function (such as responsiveness to light stimulation), or retinal structure in a subject are well known to one of skill in the art. See, e.g. Kanski's Clinical Ophthalmology: A Systematic Approach, Edition 8, Elsevier Health Sciences, 2015. Methods for measuring or assessing retinal response to light include may include detecting an electrical response of the retina to a light stimulus. This response can be detected by measuring an electroretinogram (ERG; for example full-field ERG, multifocal ERG, or ERG photostress test), visual evoked potential, or optokinetic nystagmus (see, e.g., Wester et al., Invest. Ophthalmol. Vis. Sci. 48:4542-4548, 2007). Furthermore, retinal response to light may be measured by directly detecting retinal response (for example by use of a microelectrode at the retinal surface). ERG has been extensively described by Vincent et al. Retina, 2013 January;33(1):5-12. Thus, methods of the present disclosure can be used to improve visual function, retinal function (such as responsiveness to light stimulation), retinal structure, or any other clinical symptoms or phenotypic changes associated with ocular diseases in subjects afflicted with ocular disease.

The dosage of the therapeutic formulation will vary widely, depending upon the nature of the disease, the patient's medical history, the frequency of administration, the manner of administration, the clearance of the agent from the host, and the like. The initial dose may be larger, followed by smaller maintenance doses. The dose may be administered as infrequently as weekly or biweekly, or fractionated into smaller doses and administered daily, semi-weekly, etc., to maintain an effective dosage level. In some cases, oral administration will require a higher dose than if administered intravenously. In some cases, topical administration will include application several times a day, as needed, for a number of days or weeks in order to provide an effective topical dose.

The term “carrier” refers to a diluent, adjuvant, excipient, or vehicle with which the compound is administered. Such pharmaceutical carriers can be sterile liquids, such as water and oils, including those of petroleum, animal, vegetable or synthetic origin, such as peanut oil, soybean oil, mineral oil, olive oil, sesame oil and the like. Water or aqueous solution saline solutions and aqueous dextrose and glycerol solutions are preferably employed as carriers, particularly for injectable solutions. Alternatively, the carrier can be a solid dosage form carrier, including but not limited to one or more of a binder (for compressed pills), a glidant, an encapsulating agent, a flavorant, and a colorant. Suitable pharmaceutical carriers are described in “Remington's Pharmaceutical Sciences” by E. W. Martin.

As used herein, the term “adjuvant” refers to a compound or mixture that enhances the immune response to an antigen. An adjuvant can serve as a tissue depot that slowly releases the antigen and also as a lymphoid system activator that non-specifically enhances the immune response (Hood et al., Immunology, Second Ed., 1984, Benjamin/Cummings: Menlo Park, Calif., p. 384). Often, a primary challenge with an antigen alone, in the absence of an adjuvant, will fail to elicit a humoral or cellular immune response. Adjuvants include, but are not limited to, complete Freund's adjuvant, incomplete Freund's adjuvant, saponin, mineral gels such as aluminum hydroxide, surface active substances such as lysolecithin, pluronic polyols, polyanions, peptides, oil or hydrocarbon emulsions, keyhole limpet hemocyanins, and potentially useful human adjuvants such as N-acetyl-muramyl-L-threonyl-D-isoglutamine (thr-MDP), N-acetyl-nor-muramyl-L-alanyl-D-isoglutamine, N-acetylmuramyl-L-alanyl-D-isoglutaminyl-L-alanine-2-(1′-2′-dipalmitoyl-sn-glycero-3-hydroxyphosphoryloxy)-ethylamine, and BCG (bacille Calmette-Guerin). Preferably, the adjuvant is pharmaceutically acceptable.

Vectors of the present disclosure can comprise any of a number of promoters known to the art, wherein the promoter is constitutive, regulatable or inducible, cell type specific, tissue-specific, or species specific. In addition to the sequence sufficient to direct transcription, a promoter sequence of the invention can also include sequences of other regulatory elements that are involved in modulating transcription (e.g.: enhancers, kozak sequences and introns). Many promoter/regulatory sequences useful for driving constitutive expression of a gene are available in the art and include, but are not limited to, for example, CMV (cytomegalovirus promoter), EF1a (human elongation factor 1 alpha promoter), SV40 (simian vacuolating virus 40 promoter), PGK (mammalian phosphoglycerate kinase promoter), Ubc (human ubiquitin C promoter), human beta-actin promoter, rodent beta-actin promoter, CBh (chicken beta-actin promoter), CAG (hybrid promoter contains CMV enhancer, chicken beta actin promoter, and rabbit beta-globin splice acceptor), TRE (Tetracycline response element promoter), H1 (human polymerase III RNA promoter), U6 (human U6 small nuclear promoter), and the like. Moreover, inducible and tissue specific expression of an RNA, transmembrane proteins, or other proteins can be accomplished by placing the nucleic acid encoding such a molecule under the control of an inducible or tissue specific promoter/regulatory sequence. Examples of tissue specific or inducible promoter/regulatory sequences which are useful for this purpose include, but are not limited to, the rhodopsin promoter, the MMTV LTR inducible promoter, the SV40 late enhancer/promoter, synapsin 1 promoter, ET hepatocyte promoter, GS glutamine synthase promoter and many others. Various commercially available ubiquitous as well as tissue-specific promoters can be found at http://www.invivogen.com/prom-a-list and https://www.addgene.org/

In addition, promoters which are well known in the art can be induced in response to inducing agents such as metals, glucocorticoids, tetracycline, hormones, and the like, are also contemplated for use with the invention. Thus, it will be appreciated that the present disclosure includes the use of any promoter/regulatory sequence known in the art that is capable of driving expression of the desired protein operably linked thereto.

Vectors according to the present disclosure can be transformed, transfected or otherwise introduced into a wide variety of host cells. Transfection refers to the taking up of a vector by a host cell whether or not any coding sequences are in fact expressed. Numerous methods of transfection are known to the ordinarily skilled artisan, for example, lipofectamine, calcium phosphate co-precipitation, electroporation, DEAE-dextran treatment, microinjection, viral transduction, and other methods known in the art. Transduction refers to entry of a virus into the cell and expression (e.g., transcription and/or translation) of sequences delivered by the viral vector genome. In the case of a recombinant vector, “transduction” generally refers to entry of the recombinant viral vector into the cell and expression of a nucleic acid of interest delivered by the vector genome.

In certain embodiments, the methods described herein can be utilized to treat ocular disease, neuronal disease, or improve photoreceptor function in a patient and can comprise administering to the patient an effective concentration of a composition comprising any of the recombinant AAVs described herein and a pharmaceutically acceptable carrier. In one embodiment, an effective concentration of virus is 1×10⁶−11×10¹³ GC/ml. The range of viral concentration effective for the treatment can vary depending on factors including, but not limited to specific mutation, patient's age, and other clinical parameters.

Production of recombinant AAV vectors and their use in in vitro and in vivo administration has been discussed in detail by Gray et al. (Curr Protoc Neurosci. 2011 October, Chapter:Unit 4.17).

The recombinant AAV containing the desired recombinant DNA can be formulated into a pharmaceutical composition intended for subretinal or intravitreal injection. Such formulation involves the use of a pharmaceutically and/or physiologically acceptable vehicle or carrier, particularly one suitable for administration to the eye, e.g., by subretinal injection, such as buffered saline or other buffers, e.g., HEPES, to maintain pH at appropriate physiological levels, and, optionally, other medicinal agents, pharmaceutical agents, stabilizing agents, buffers, carriers, adjuvants, diluents, etc. For injection, the carrier will typically be a liquid. Exemplary physiologically acceptable carriers include sterile, pyrogen-free water and sterile, pyrogen-free, phosphate buffered saline.

In one embodiment, the carrier is an isotonic sodium chloride solution. In another embodiment, the carrier is balanced salt solution. In one embodiment, the carrier includes tween. If the virus is to be stored long-term, it may be frozen in the presence of glycerol or Tween-20. In another embodiment, the pharmaceutically acceptable carrier comprises a surfactant, such as perfluorooctane (Perfluoron liquid). In certain embodiments, the pharmaceutical composition described above is administered to the subject by subretinal injection. In other embodiments, the pharmaceutical composition is administered by intravitreal injection. Other forms of administration that may be useful in the methods described herein include, but are not limited to, direct delivery to a desired organ (e.g., the eye), oral, inhalation, intranasal, intratracheal, intravenous, intramuscular, subcutaneous, intradermal, and other parental routes of administration. Additionally, routes of administration may be combined, if desired.

In some embodiments, route of administration is subretinal injection or intravitreal injection.

### Inhibitors of VHL

By “VHL,” “VHL,” “Vhl,” “Vhl” is meant to include the DNA, RNA, mRNA, cDNA, recombinant DNA or RNA, or the protein arising from the Vhl gene or VHL interactors. The human reference sequences can be found at: HGNC: 12687 Entrez Gene: 7428 Ensembl: ENSG00000134086 OMIM: 608537 UniProtKB: P40337. The mouse nucleotide sequence can be found at NCBI Gene: 22346.

Von Hippel-Lindau syndrome (VHL) is a dominantly inherited familial cancer syndrome predisposing to a variety of malignant and benign tumors. A germline mutation of this gene is the basis of familial inheritance of VHL syndrome. The protein encoded by this gene is a component of the protein complex that includes elongin B, elongin C, and cullin-2, and possesses ubiquitin ligase E3 activity. This protein is involved in the ubiquitination and degradation of hypoxia-inducible-factor (HIF), which is a transcription factor that activates glycolysis via GLUT1, GLUT3, PDK, and NADPH. Alternatively spliced transcript variants encoding distinct isoforms have been observed. Diseases associated with VHL include Von Hippel-Lindau Syndrome and Erythrocytosis, Familial, 2. Among its related pathways are Hypoxic and oxygen homeostasis regulation of HIF-1-alpha and Immune System. GO annotations related to this gene include enzyme binding and ubiquitin-protein transferase activity. An important paralog of this gene is VHLL. UniProtKB/Swiss-Prot for VHL Gene: VHL_HUMAN: P40337. It is involved in the ubiquitination and subsequent proteasomal degradation via the von Hippel-Lindau ubiquitination complex. Seems to act as a target recruitment subunit in the E3 ubiquitin ligase complex and recruits hydroxylated hypoxia-inducible factor (HIF) under normoxic conditions. It is further involved in transcriptional repression through interaction with HIF1A, HIF1AN and histone deacetylases. Ubiquitinates, in an oxygen-responsive manner, ADRB2.

It is noted that as used herein VHL can refer to the gene or the protein encoded for by the gene, as appropriate in the specific context utilized. Additionally, in certain contexts, the reference will be to the mouse gene or protein, and in others the human gene or protein as appropriate in the specific context.

Any isoform of any VHL may be inhibited by the present inhibitors. Isoforms of VHL include, but are not limited to: human VHL isoform 1 (e.g., NCBI Reference Sequence: NM_000551 for mRNA, NCBI Reference Sequence: NP_000542 for protein), human VHL isoform 2 (e.g., NCBI Reference Sequence: NM_198156 for mRNA, NCBI Reference Sequence: NP_937799 for protein), and human VHL isoform 3 (e.g., NCBI Reference Sequence: NM_001354723 for mRNA, NCBI Reference Sequence: NP_001341652 for protein). Murine VHL may be NCBI Reference Sequence NM_009507 for mRNA, and NCBI Reference Sequence NP_033533 for protein.

The present inhibitors may target the wild-type or mutant form of VHL.

As used herein, the term “inhibitor” refers to agents capable of down-regulating or otherwise decreasing or suppressing the amount/level and/or activity of VHL.

The mechanism of inhibition may be at the genetic level (e.g., interference with or inhibit expression, transcription or translation, etc.) or at the protein level (e.g., binding, competition, etc.).

A wide variety of suitable inhibitors may be employed, guided by art-recognized criteria such as efficacy, toxicity, stability, specificity, half-life, etc.

### Small Molecule Inhibitors

As used herein, the term “small molecules” encompasses molecules other than proteins or nucleic acids without strict regard to size. Non-limiting examples of small molecules that may be used according to the methods and compositions of the present invention include, small organic molecules, peptide-like molecules, peptidomimetics, carbohydrates, lipids or other organic (carbon containing) or inorganic molecules.

In one embodiment, the inhibitor of VHL is a compound of Formula (I):

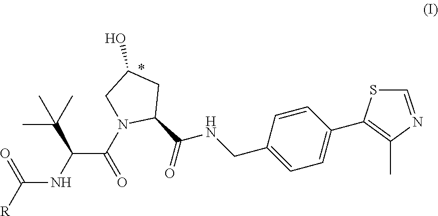

For example, the inhibitor of VHL may be VH298 ((2S,4R)-1-((S)-2-(1-cyanocyclopropanecarboxamido)-3,3-dimethylbutanoyl)-4-hydroxy-N-(4-(4-methylthiazol-5-yl)benzyl)pyrrolidine-2-carboxamide), VH032 ((2S,4R)-1-((S)-2-acetamido-3,3-dimethylbutanoyl)-4-hydroxy-N-(4-(4-methylthiazol-5-yl)benzyl)pyrrolidine-2-carboxamide), or VH125. Frost et al., Potent and selective chemical probe of hypoxic signaling downstream of HIF-α hydroxylation via VHL inhibition, Nature Communications, 2016, 7, 13312. Galdeano et al. Structure-guided design and optimization of small molecules targeting the protein-protein interaction between the von Hippel-Lindau (VHL) E3 ubiquitin ligase and the hypoxia inducible factor (HIF) alpha subunit with in vitro nanomolar affinities. J. Med. Chem. 57, 8657-8663 (2014).

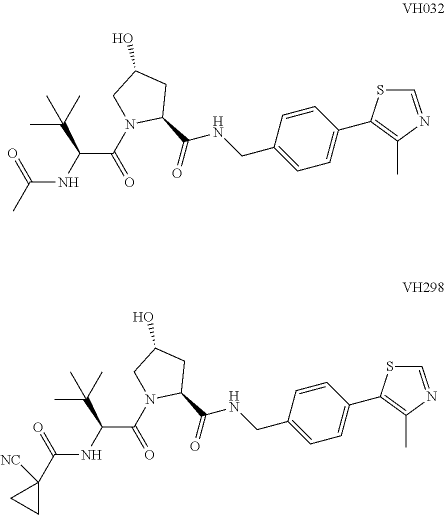 [SMILES: CC(=O)N[C@H](C(=O)N1C[C@H](O)C[C@H]1C(=O)NCc1ccc(-c2scnc2C)cc1)C(C)(C)C ; Cc1ncsc1-c1ccc(CNC(=O)[C@@H]2C[C@@H](O)CN2C(=O)[C@@H](NC(=O)C2(C#N)CC2)C(C)(C)C)cc1]

The inhibitor may be any of the compounds described in Soares et al., Group-Based Optimization of Potent and Cell-Active Inhibitors of the von Hippel-Lindau (VHL) E3 Ubiquitin Ligase: Structure-Activity Relationships Leading to the Chemical Probe (2S,4R)-1-((S)-2-(1-Cyanocyclopropanecarboxamido)-3,3-dimethylbutanoyl)-4-hydroxy-N-(4-(4-methylthiazol-5-yl)benzyl)pyrrolidine-2-carboxamide (VH298), J Med Chem. 2018 Jan 25;61(2):599-618.

### Endonucleases

Methods for modification of genomic DNA are well known in the art. For example, methods may use a DNA digesting agent to modify the DNA by either the non-homologous end joining DNA repair pathway (NHEJ) or the homology directed repair (HDR) pathway. The term “DNA digesting agent” refers to an agent that is capable of cleaving bonds (i.e. phosphodiester bonds) between the nucleotide subunits of nucleic acids.

In one embodiment, the DNA digesting agent is a nuclease. Nucleases are enzymes that hydrolyze nucleic acids. Nucleases may be classified as endonucleases or exonucleases. An endonuclease is any of a group of enzymes that catalyze the hydrolysis of bonds between nucleic acids in the interior of a DNA or RNA molecule. An exonuclease is any of a group of enzymes that catalyze the hydrolysis of single nucleotides from the end of a DNA or RNA chain. Nucleases may also be classified based on whether they specifically digest DNA or RNA. A nuclease that specifically catalyzes the hydrolysis of DNA may be referred to as a deoxyribonuclease or DNase, whereas a nuclease that specifically catalyses the hydrolysis of RNA may be referred to as a ribonuclease or an RNase. Some nucleases are specific to either single-stranded or double-stranded nucleic acid sequences. Some enzymes have both exonuclease and endonuclease properties. In addition, some enzymes are able to digest both DNA and RNA sequences.

VHL may be inhibited by using a sequence-specific endonuclease that target the gene encoding VHL.

Non-limiting examples of the endonucleases include a zinc finger nuclease (ZFN), a ZFN dimer, a ZFNickase, a transcription activator-like effector nuclease (TALEN), or a RNA-guided DNA endonuclease (e.g., CRISPR/Cas9). Meganucleases are endonucleases characterized by their capacity to recognize and cut large DNA sequences (12 base pairs or greater). Any suitable meganuclease may be used in the present methods to create double-strand breaks in the host genome, including endonucleases in the LAGLIDADG and PI-Sce family.

The sequence-specific endonuclease of the methods and compositions described herein can be engineered, chimeric, or isolated from an organism. Endonucleases can be engineered to recognize a specific DNA sequence, by, e.g., mutagenesis. Seligman et al. (2002) Mutations altering the cleavage specificity of a homing endonuclease, Nucleic Acids Research 30: 3870-3879. Combinatorial assembly is a method where protein subunits form different enzymes can be associated or fused. Arnould et al. (2006) Engineering of large numbers of highly specific homing endonucleases that induce recombination to novel DNA targets, Journal of Molecular Biology 355: 443-458. These two approaches, mutagenesis and combinatorial assembly, may be combined to produce an engineered endonuclease with desired DNA recognition sequence.

The sequence-specific nuclease can be introduced into the cell in the form of a protein or in the form of a nucleic acid encoding the sequence-specific nuclease, such as an mRNA or a cDNA. Nucleic acids can be delivered to a bacterial cell by transformation, e.g., heat shock, electroporation, etc. In one embodiment, bacterial cells are incubated in a solution containing divalent cations (e.g., calcium chloride) under cold conditions, before being exposed to a heat pulse (heat shock).

One example of a sequence-specific nuclease system that can be used with the methods and compositions described herein includes the CRISPR system (Wiedenheft, B. et al. Nature 482, 331-338 (2012); Jinek, M. et al. Science 337, 816-821 (2012); Mali, P. et al. Science 339, 823-826 (2013); Cong, L. et al. Science 339, 819-823 (2013)). The CRISPR (Clustered Regularly interspaced Short Palindromic Repeats) system exploits RNA-guided DNA-binding and sequence-specific cleavage of target DNA. The guide RNA/Cas combination confers site specificity to the nuclease. A single guide RNA (sgRNA) contains about 20 nucleotides that are complementary to a target genomic DNA sequence upstream of a genomic PAM (protospacer adjacent motifs) site (NGG) and a constant RNA scaffold region. The Cas (CRISPR-associated) protein binds to the sgRNA and the target DNA to which the sgRNA binds and introduces a double-strand break in a defined location upstream of the PAM site. Cas9 harbors two independent nuclease domains homologous to HNH and RuvC endonucleases, and by mutating either of the two domains, the Cas9 protein can be converted to a nickase that introduces single-strand breaks (Cong, L. et al. Science 339, 819-823 (2013)). It is specifically contemplated that the methods and compositions of the present disclosure can be used with the single- or double-strand-inducing version of Cas9, as well as with other RNA-guided DNA nucleases, such as other bacterial Cas9-like systems. The sequence-specific nuclease of the present methods and compositions described herein can be engineered, chimeric, or isolated from an organism. The nuclease can be introduced into the cell in form of a DNA, mRNA and protein. The applications of the CRISPR/Cas system to inhibiting or downregulating Vhl are easily adapted.

In one embodiment, the methods of the present disclosure comprise using one or more sgRNAs to remove, or suppress a glycosis regulator, such as Vhl. In another embodiment, one sgRNA(s) is used to remove, or suppress a glycosis regulator, Vhl disease- related gene. In yet further embodiment, two or more sgRNA(s) are used to remove, or suppress an autosomal dominant disease-related gene.

In one embodiment, the DNA digesting agent can be a site-specific nuclease. In another embodiment, the site-specific nuclease may be a Cas-family nuclease. In a more specific embodiment, the Cas nuclease may be a Cas9 nuclease.

In one embodiment, Cas protein may be a functional derivative of a naturally occurring Cas protein.

In addition to well characterized CRISPR-Cas system, a new CRISPR enzyme, called Cpfl (Cas protein 1 of PreFran subtype) has recently been described (Zetsche et al. Cell. pii: S0092-8674(15)01200-3. doi: 10.1016/j.cell.2015.09.038 (2015)). Cpfl is a single RNA-guided endonuclease that lacks tracrRNA, and utilizes a T-rich protospacer-adjacent motif. The authors demonstrated that Cpf1 mediates strong DNA interference with characteristics distinct from those of Cas9. Thus, in one embodiment of the present invention, CRISPR-Cpf1 system can be used to cleave a desired region within the targeted gene.

In further embodiment, the DNA digesting agent is a transcription activator-like effector nuclease (TALEN). TALENs are composed of a TAL effector domain that binds to a specific nucleotide sequence and an endonuclease domain that catalyzes a double strand break at the target site (PCT Patent Publication No. WO2011072246; Miller et al., Nat. Biotechnol. 29, 143-148 (2011); Cermak et al., Nucleic Acid Res. 39, e82 (2011)). Sequence-specific endonucleases may be modular in nature, and DNA binding specificity is obtained by arranging one or more modules. Bibikova et al., Mol. Cell. Biol. 21, 289-297 (2001). Boch et al., Science 326, 1509-1512 (2009).

ZFNs can be composed of two or more (e.g., 2-8, 3-6, 6-8, or more) sequence-specific DNA binding domains (e.g., zinc finger domains) fused to an effector endonuclease domain (e.g., the FokI endonuclease). Porteus et al., Nat. Biotechnol. 23, 967-973 (2005). Kim et al. (2007) Hybrid restriction enzymes: Zinc finger fusions to Fok I cleavage domain, Proceedings of the National Academy of Sciences of USA, 93: 1156-1160. U.S. Pat. No. 6,824,978. PCT Publication Nos. WO1995/09233 and WO1994018313.

In one embodiment, the DNA digesting agent is a site-specific nuclease of the group or selected from the group consisting of omega, zinc finger, TALE, and CRISPR/Cas.

The sequence-specific endonuclease of the methods and compositions described here can be engineered, chimeric, or isolated from an organism. Endonucleases can be engineered to recognize a specific DNA sequence, by, e.g., mutagenesis. Seligman et al. (2002) Mutations altering the cleavage specificity of a homing endonuclease, Nucleic Acids Research 30: 3870-3879. Combinatorial assembly is a method where protein subunits form different enzymes can be associated or fused. Arnould et al. (2006) Engineering of large numbers of highly specific homing endonucleases that induce recombination to novel DNA targets, Journal of Molecular Biology 355: 443-458. In certain embodiments, these two approaches, mutagenesis and combinatorial assembly, can be combined to produce an engineered endonuclease with desired DNA recognition sequence.

The sequence-specific nuclease can be introduced into the cell in the form of a protein or in the form of a nucleic acid encoding the sequence-specific nuclease, such as an mRNA or a cDNA. Nucleic acids can be delivered as part of a larger construct, such as a plasmid or viral vector, or directly, e.g., by electroporation, lipid vesicles, viral transporters, microinjection, and biolistics. Similarly, the construct containing the one or more transgenes can be delivered by any method appropriate for introducing nucleic acids into a cell.

Single guide RNA(s) used in the methods of the present disclosure can be designed so that they direct binding of the Cas-sgRNA complexes to pre-determined cleavage sites in a genome. In one embodiment, the cleavage sites may be chosen so as to release a fragment or sequence that contains a region of autosomal dominant disease-related gene. In further embodiment, the cleavage sites may be chosen so as to release a fragment or sequence that contains a region of genes encoding glycosis regulators, e.g. Vhl.

For Cas family enzyme (such as Cas9) to successfully bind to DNA, the target sequence in the genomic DNA should be complementary to the sgRNA sequence and must be immediately followed by the correct protospacer adjacent motif or “PAM” sequence. “Complementarity” refers to the ability of a nucleic acid to form hydrogen bond(s) with another nucleic acid sequence by either traditional Watson-Crick or other non-traditional types. A percent complementarity indicates the percentage of residues in a nucleic acid molecule, which can form hydrogen bonds (e.g., Watson-Crick base pairing) with a second nucleic acid sequence. Full complementarity is not necessarily required, provided there is sufficient complementarity to cause hybridization and promote formation of a CRISPR complex. A target sequence may comprise any polynucleotide, such as DNA or RNA polynucleotides. The Cas9 protein can tolerate mismatches distal from the PAM, however, mismatches within the 12 base pairs (bps) of sequence next to the PAM sequence can dramatically decrease the targeting efficiency. The PAM sequence is present in the DNA target sequence but not in the sgRNA sequence. Any DNA sequence with the correct target sequence followed by the PAM sequence will be bound by Cas9. The PAM sequence varies by the species of the bacteria from which Cas9 was derived. The most widely used CRISPR system is derived from S. pyogenes and the PAM sequence is NGG located on the immediate 3′ end of the sgRNA recognition sequence. The PAM sequences of CRISPR systems from exemplary bacterial species include: Streptococcus pyogenes (NGG), Neisseria meningitidis (NNNNGATT), Streptococcus thermophilus (NNAGAA) and Treponema denticola (NAAAAC).

sgRNA(s) used in the present disclosure can be between about 5 and 100 nucleotides long, or longer (e.g., 5, 6, 7, 8, 9, 10, 11, 12, 13, 14, 15, 16, 17, 18, 19, 20, 21, 22, 23, 24, 25, 26, 27, 28, 29, 30, 31 , 32, 33, 34, 35, 36, 37, 38, 39, 40, 41, 42, 43, 44, 45, 46, 47, 48, 49, 50, 51 , 52, 53, 54, 55, 56, 57, 58, 59 60, 61, 62, 63, 63, 64, 65, 66, 67, 68, 69, 70, 71, 72, 73, 74, 75, 76, 77, 78, 79, 80, 81 , 82, 83, 84, 85, 86, 87, 88, 89, 90, 91 92, 93, 94, 95, 96, 97, 98, 99, or 100 nucleotides in length, or longer). In one embodiment, sgRNA(s) can be between about 15 and about 30 nucleotides in length (e.g., about 15-29, 15-26, 15-25; 16-30, 16-29, 16-26, 16-25; or about 18-30, 18-29, 18-26, or 18-25 nucleotides in length).

To facilitate sgRNA design, many computational tools have been developed (See Prykhozhij et al. (PLoS ONE, 10(3): (2015)); Zhu et al. (PLoS ONE, 9(9) (2014)); Xiao et al. (Bioinformatics. Jan 21 (2014)); Heigwer et al. (Nat Methods, 11(2): 122-123 (2014)). Methods and tools for guide RNA design are discussed by Zhu (Frontiers in Biology, 10 (4) pp 289-296 (2015)), which is incorporated by reference herein. Additionally, there is a publically available software tool that can be used to facilitate the design of sgRNA(s) (http://www.genscript.com/gRNA-design-tool.html).

### Inhibitory Nucleic Acids that Hybridize to VHL

In certain embodiments, the VHL inhibitor used in the present methods and compositions is a polynucleotide that reduces expression of VHL. Thus, the method involves administering an effective amount of a polynucleotide that specifically targets nucleotide sequence(s) encoding VHL. The polynucleotides reduce expression of VHL, to yield reduced levels of the gene product (the translated polypeptide).

The nucleic acid target of the polynucleotides (e.g., antisense oligonucleotides, and ribozymes) may be any location within the gene or transcript of VHL.

Any number of means for inhibiting VHL activity or gene expression can be used in the methods of the invention. For example, a nucleic acid molecule complementary to at least a portion of a human VHL encoding nucleic acid can be used to inhibit Vhl gene expression. Means for inhibiting gene expression using short RNA molecules, for example, are known. Among these are short interfering RNA (siRNA), small temporal RNAs (stRNAs), and micro-RNAs (miRNAs). Short interfering RNAs silence genes through an mRNA degradation pathway, while stRNAs and miRNAs are approximately 21 or 22 nt RNAs that are processed from endogenously encoded hairpin-structured precursors, and function to silence genes via translational repression. See, e.g., McManus et al., RNA, 8(6):842-50 (2002); Morris et al., Science, 305(5688):1289-92 (2004); He and Hannon, Nat Rev Genet. 5(7):522-31 (2004).

“RNA interference, or RNAi” is a form of post-transcriptional gene silencing (“PTGS”), and describes effects that result from the introduction of double-stranded RNA into cells (reviewed in Fire, A. Trends Genet 15:358-363 (1999); Sharp, P. Genes Dev 13:139-141 (1999); Hunter, C. Curr Biol 9:R440-R442 (1999); Baulcombe. D. Curr Biol 9:R599-R601 (1999); Vaucheret et al. Plant J 16: 651-659 (1998)). RNA interference, commonly referred to as RNAi, offers a way of specifically inactivating a cloned gene, and is a powerful tool for investigating gene function.

The active agent in RNAi is a long double-stranded (antiparallel duplex) RNA, with one of the strands corresponding or complementary to the RNA which is to be inhibited. The inhibited RNA is the target RNA. The long double stranded RNA is chopped into smaller duplexes of approximately 20 to 25 nucleotide pairs, after which the mechanism by which the smaller RNAs inhibit expression of the target is largely unknown at this time. While RNAi was shown initially to work well in lower eukaryotes, for mammalian cells, it was thought that RNAi might be suitable only for studies on the oocyte and the preimplantation embryo.

More recently, it was shown that RNAi would work in human cells if the RNA strands were provided as pre-sized duplexes of about 19 nucleotide pairs, and RNAi worked particularly well with small unpaired 3′ extensions on the end of each strand (Elbashir et al. Nature 411: 494-498 (2001)). In this report, “short interfering RNA” (siRNA, also referred to as small interfering RNA) were applied to cultured cells by transfection in oligofectamine micelles. These RNA duplexes were too short to elicit sequence-nonspecific responses like apoptosis, yet they efficiently initiated RNAi. Many laboratories then tested the use of siRNA to knock out target genes in mammalian cells. The results demonstrated that siRNA works quite well in most instances.

Software programs for predicting siRNA sequences to inhibit the expression of a target protein are commercially available and find use. One program, siDESIGN from Dharmacon, Inc. (Lafayette, Colo.), permits predicting siRNAs for any nucleic acid sequence, and is available on the internet at dharmacon.com. Programs for designing siRNAs are also available from others, including Genscript (available on the internet at genscript.com/ssl-bin/app/rnai) and, to academic and non-profit researchers, from the Whitehead Institute for Biomedical Research found on the worldwide web at “jura.wi.mit.edu/pubint/http://iona.wi.mit.edu/siRNAext/.”

Any suitable viral knockdown system could be utilized for decreasing Vhl mRNA levels—including AAV, lentiviral vectors, or other suitable vectors that are capable of being targeted specifically to the liver. (See Zuckerman and Davis 2015).

Additionally, specifically targeted delivery of shVhl mRNA or other Vhl blocking molecule (nucleic acid, peptide, or small molecule) could be delivered by targeted liposome, nanoparticle or other suitable means.

As described herein we provide methods as well as one or more agents/compounds that silence or inhibit Vhl/VHL, for the treatment, prophylaxis or alleviation of degenerative eye conditions including RP AMD, glaucoma, and related conditions, as well as neurodegenerative conditions described herein, or predisposition to such conditions.

RNA interference (RNAi) is a method of post transcriptional gene silencing (PTGS) induced by the direct introduction of double-stranded RNA (dsRNA) and has emerged as a useful tool to knock out expression of specific genes in a variety of organisms. RNAi is described by Fire et al., Nature 391:806-811 (1998). Other methods of PTGS are known and include, for example, introduction of a transgene or virus. Generally, in PTGS, the transcript of the silenced gene is synthesised but does not accumulate because it is rapidly degraded. Methods for PTGS, including RNAi are described, for example, in the Ambion.com world wide web site, in the directory “/hottopics/”, in the “rnai” file.

Suitable methods for RNAi in vitro are described herein. One such method involves the introduction of siRNA (small interfering RNA). Current models indicate that these 21-23 nucleotide dsRNAs can induce PTGS. Methods for designing effective siRNAs are described, for example, in the Ambion web site described above. RNA precursors such as Short Hairpin RNAs (shRNAs) can also be encoded by all or a part of the Vhl nucleic acid sequence.

Alternatively, double-stranded (ds) RNA is a powerful way of interfering with gene expression in a range of organisms that has recently been shown to be successful in mammals (Wianny and Zernicka-Goetz, 2000, Nat Cell Biol 2:70-75). Double stranded RNA corresponding to the sequence of a Vhl polynucleotide can be introduced into or expressed in oocytes and cells of a candidate organism to interfere with Vhl activity.

Non-limiting examples of micro-RNAs (miRNAs) that may inhibit VHL include microRNA-92 (miR-92). Guo et al., MIR-92 stimulates VEGF by inhibiting von Hippel-Lindau gene product in epithelial ovarian cancer, J. Biol. Regul. Homeost. Agents 31 (3), 615-624 (2017).

Vhl gene expression may also be modulated by introducing peptides or small molecules which inhibit gene expression or functional activity. Thus, compounds identified by the assays described herein as binding to or modulating, such as down-regulating, the amount, activity or expression of VHL polypeptide may be administered to target cells to prevent the function of VHL polypeptide. Such a compound may be administered along with a pharmaceutically acceptable carrier in an amount effective to down-regulate expression or activity VHL, or by activating or down-regulating a second signal which controls VHL expression, activity or amount, and thereby alleviating the abnormal condition.

Alternatively, gene therapy may be employed to control the endogenous production of Vhl by the relevant cells such as neuronal cells or photoreceptor cells, i.e., rod and cone cells in the subject. For example, a polynucleotide encoding a Vhl siRNA or a portion of this may be engineered for expression in a replication defective retroviral vector, as discussed below. The retroviral expression construct may then be isolated and introduced into a packaging cell transduced with a retroviral plasmid vector containing RNA encoding an anti-Vhl siRNA such that the packaging cell now produces infectious viral particles containing the sequence of interest. These producer cells may be administered to a subject for engineering cells in vivo and regulating expression of the VHL polypeptide in vivo. For overview of gene therapy, see Chapter 20, Gene Therapy and other Molecular Genetic-based Therapeutic Approaches, (and references cited therein) in Human Molecular Genetics, T Strachan and A P Read, BIOS Scientific Publishers Ltd (1996).

In some embodiments, the level of VHL is decreased in a desired target cell such as a neuronal cell or the vitreous. Furthermore, in such embodiments, treatment may be targeted to, or specific to, desired target cell such as a neuronal cell or the vitreous. The expression of VHL may be specifically decreased only in the desired target cell such as a neuronal cell or the vitreous (i.e., those cells which are predisposed to the condition, or exhibiting the disease already), and not substantially in other non-diseased cells. In these methods, expression of VHL may not be substantially reduced in other cells, i.e., cells which are not desired target cells. Thus, in such embodiments, the level of VHL, remains substantially the same or similar in non-target cells in the course of or following treatment.

Alternately, one may administer the viral vectors, RNAi, shRNA or other Vhl inhibitor, or related compounds in a local rather than systemic manner, for example, via injection of directly into the desired target site, often in a depot or sustained release formulation. Furthermore, one may administer the composition in a targeted drug delivery system, for example, in a liposome coated with a tissue-specific antibody, targeting, for example, specific neurons, or the vitreous, and more specifically hepatocytes. The liposomes will be targeted to and taken up selectively by the desired tissue. Also included in a targeted drug delivery system is nanoparticle specific delivery of the viral vectors, RNAi, shRNA or other Vhl inhibitors, alone or in combination. A summary of various delivery methods and techniques of siRNA administration in ongoing clinical trials is provided in Zuckerman and Davis 2015; Nature Rev. Drug Discovery, Vol. 14: 843-856, December 2015.

The inhibitory nucleic acids may be an antisense nucleic acid sequence that is complementary to a target region within the mRNA of VHL. The antisense polynucleotide may bind to the target region and inhibit translation. The antisense oligonucleotide may be DNA or RNA, or comprise synthetic analogs of ribo-deoxynucleotides. Thus, the antisense oligonucleotide inhibits expression of VHL.

An antisense oligonucleotide can be, for example, about 7, 10, 15, 20, 25, 30, 35, 40, 45, 50, 55, 60, 65, 70, 75, 80, or more nucleotides in length.

The antisense nucleic acid molecules of the invention may be administered to a subject, or generated in situ such that they hybridize with or bind to the mRNA of VHL.

The administration regimen depends on several factors, including the serum or tissue turnover rate of the therapeutic composition, the level of symptoms, and the accessibility of the target cells in the biological matrix. Preferably, the administration regimen delivers sufficient therapeutic composition to effect improvement in the target disease state, while simultaneously minimizing undesired side effects. Accordingly, the amount of biologic delivered depends in part on the particular therapeutic composition and the severity of the condition being treated.

In preferred embodiments, route of administration is subretinal injection or intravitreal injection.

### Ribozyme

The inhibitor may be a ribozyme that inhibits expression of the VHL gene.

Ribozymes can be chemically synthesized and structurally modified to increase their stability and catalytic activity using methods known in the art. Ribozyme encoding nucleotide sequences can be introduced into host cells through gene-delivery mechanisms known in the art.

### Antibodies

The present inhibitors can be an antibody or antigen-binding portion thereof that is specific to VHL.

The antibody or antigen-binding portion thereof may be the following: (a) a whole immunoglobulin molecule; (b) an scFv; (c) a Fab fragment; (d) an F(ab′)2; and (e) a disulfide linked Fv. The antibody or antigen-binding portion thereof may be monoclonal, polyclonal, chimeric and humanized. The antibodies may be murine, rabbit or human/humanized antibodies.

### Combination Therapy

The VHL inhibitor may be administered alone or in combination with a second treatment, such as administration of one or more second agents (different from the present VHL inhibitors), and surgeries.

Non-limiting examples of the second treatments including, treatment with vitamin A, docosahexaenoic acid (DHA), lutein, zeaxanthin, bevacizumab, ranibizumab, pegaptanib, aflibercept, an optic prosthetic device, a gene therapy, a retinal implant (e.g., Argus retinal prosthesis) and/or a retinal sheet transplantation, laser coagulation, photodynamic therapy, and cataract surgery.

The second agents that may be used with the present VHL inhibitor also include, but are not limited to, prostaglandin analogs (e.g., latanoprost, bimatoprost and travoprost), topical beta-adrenergic receptor antagonists (e.g., timolol, levobunolol, and betaxolol), alpha2-adrenergic agonists (e.g., such as brimonidine and apraclonidine), less-selective alpha agonists (e.g., epinephrine), miotic agents (parasympathomimetics, e.g., pilocarpine, and echothiophate), carbonic anhydrase inhibitors (e.g., dorzolamide, brinzolamide, and acetazolamide).

The second treatments that may be used with the present VHL inhibitor include, but are not limited to, laser surgery (e.g., Argon laser trabeculoplasty (ALT), selective laser trabeculoplasty (SLT), Nd:YAG laser peripheral iridotomy (LPI), diode laser cycloablation, traditional laser trabeculoplasty), canaloplasty, trabeculectomy, glaucoma drainage implants, and laser-assisted nonpenetrating deep sclerectomy.

Combinations may be administered either concomitantly, e.g., as an admixture, separately but simultaneously or concurrently; or sequentially. This includes presentations in which the combined agents are administered together as a therapeutic mixture, and also procedures in which the combined agents are administered separately but simultaneously, e.g., as through separate intravenous lines into the same individual. Administration “in combination” further includes the separate administration of one of the compounds or agents given first, followed by the second.

This may be achieved by administering a pharmaceutical composition that includes both agents, or by administering two pharmaceutical compositions, at the same time or within a short time period.

In certain embodiments, the combination of the present VHL inhibitor and the second treatment produces an additive or synergistic effect (i.e., greater than additive effect) in treating a disorder as discussed herein, compared to the effect of the VHL inhibitor alone or the second treatment alone.

As used herein, the term “synergy” (or “synergistic”) means that the effect achieved with the methods and combinations of the combination therapy is greater than the sum of the effects that result from using the individual agents alone, e.g., using the VHL inhibitor alone and the second treatment alone. For example, the effect achieved with the combination of the VHL inhibitor and the second treatment is about 1.1 fold, about 1.2 fold, about 1.3 fold, about 1.4 fold, about 1.5 fold, about 1.6 fold, about 1.7 fold, about 1.8 fold, about 1.9 fold, about 2 fold, about 2.5 fold, about 3 fold, about 3.5 fold, about 4 fold, about 4.5 fold, about 5 fold, about 5.5 fold, about 6 fold, about 6.5 fold, about 7 fold, about 8 fold, about 9 fold, about 10 fold, about 12 fold, about 15 fold, about 20 fold, about 25 fold, about 30 fold, about 50 fold, about 100 fold, at least about 1.2 fold, at least about 1.5 fold, at least about 2 fold, at least about 2.5 fold, at least about 3 fold, at least about 3.5 fold, at least about 4 fold, at least about 4.5 fold, at least about 5 fold, at least about 5.5 fold, at least about 6 fold, at least about 6.5 fold, at least about 7 fold, at least about 8 fold, at least about 9 fold, at least about 10 fold, of the sum of the effects that result from using the VHL inhibitor alone or the second treatment alone.

Synergistic effects of the combination may also be evidenced by additional, novel effects that do not occur when either agent is administered alone, or by reduction of adverse side effects when either agent is administered alone.

In one embodiment, advantageously, such synergy provides greater efficacy at the same doses, and/or lower side effects.

For sequential administration, either a VHL inhibitor is administered first and then a second treatment, or the second treatment is administered first and then a VHL inhibitor. In embodiments where the VHL inhibitor and the second treatment are administered separately, administration of a first agent can precede administration of a second agent by seconds, minutes, hours, days, or weeks. The time difference in non-simultaneous administrations may be greater than 1 minute, and can be, for example, precisely, at least, up to, or less than 5 minutes, 10 minutes, 15 minutes, 30 minutes, 45 minutes, 60 minutes, 2 hours, 3 hours, 6 hours, 9 hours, 12 hours, 24 hours, 36 hours, or 48 hours, or more than 48 hours. The two or more agents can be administered within minutes of each other or within about 0.5, about 1, about 2, about 3, about 4, about 6, about 9, about 12, about 15, about 18, about 24, or about 36 hours of each other or within about 1, 2, 3, 4, 5, 6, 7, 8, 9, 10, 12, 14 days of each other or within about 2, 3, 4, 5, 6, 7, 8, 9, or 10 weeks of each other. In some cases, longer intervals are possible.

The present disclosure may provide for a pharmaceutical composition comprising a first amount of a VHL inhibitor and a second amount of a second agent. The combination of the first amount of a VHL inhibitor and the second amount of the second agent may produce a synergistic effect on a bacterial infection compared to the effect of the first amount of the VHL inhibitor alone or the effect of the second amount of the second agent alone.

The amount of a VHL inhibitor or the amount of the second agent that may be used in the combination therapy may be a therapeutically effective amount, a sub-therapeutically effective amount or a synergistically effective amount.

The VHL inhibitor, and/or the second agent may be present in the pharmaceutical composition in an amount ranging from about 0.005% (w/w) to about 100% (w/w), from about 0.01% (w/w) to about 90% (w/w), from about 0.1% (w/w) to about 80% (w/w), from about 1% (w/w) to about 70% (w/w), from about 10% (w/w) to about 60% (w/w), from about 0.01% (w/w) to about 15% (w/w), or from about 0.1% (w/w) to about 20% (w/w).

The VHL inhibitor and the second agent may be present in two separate pharmaceutical compositions to be used in a combination therapy.

The effective amount of the VHL inhibitor or the second agent for the combination therapy may be less than, equal to, or greater than when the agent is used alone.

### Pharmaceutical Compositions

The present agents or pharmaceutical compositions may be administered by any route, including, without limitation, oral, transdermal, ocular, intraperitoneal, intravenous, ICV, intracisternal injection or infusion, subcutaneous, implant, sublingual, subcutaneous, intramuscular, intravenous, rectal, mucosal, ophthalmic, intrathecal, intra-articular, intra-arterial, sub-arachinoid, bronchial and lymphatic administration. The present composition may be administered parenterally or systemically.

The pharmaceutical compositions of the present invention can be, e.g., in a solid, semi-solid, or liquid formulation. Intranasal formulation can be delivered as a spray or in a drop; inhalation formulation can be delivered using a nebulizer or similar device; topical formulation may be in the form of gel, ointment, paste, lotion, cream, poultice, cataplasm, plaster, dermal patch aerosol, etc.; transdermal formulation may be administered via a transdermal patch or iontorphoresis. Compositions can also take the form of tablets, pills, capsules, semisolids, powders, sustained release formulations, solutions, emulsions, suspensions, elixirs, aerosols, chewing bars or any other appropriate compositions.

The composition may be administered locally via implantation of a membrane, sponge, or another appropriate material on to which the desired molecule has been absorbed or encapsulated. Where an implantation device is used, the device may be implanted into any suitable tissue or organ, and delivery of the desired molecule may be via diffusion, timed release bolus, or continuous administration.

To prepare such pharmaceutical compositions, one or more of compound of the present invention may be mixed with a pharmaceutical acceptable excipient, e.g., a carrier, adjuvant and/or diluent, according to conventional pharmaceutical compounding techniques.

Pharmaceutically acceptable carriers that can be used in the present compositions encompass any of the standard pharmaceutical carriers, such as a phosphate buffered saline solution, water, and emulsions, such as an oil/water or water/oil emulsion, and various types of wetting agents. The compositions can additionally contain solid pharmaceutical excipients such as starch, cellulose, talc, glucose, lactose, sucrose, gelatin, malt, rice, flour, chalk, silica gel, magnesium stearate, sodium stearate, glycerol monostearate, sodium chloride, dried skim milk and the like. Liquid and semisolid excipients may be selected from glycerol, propylene glycol, water, ethanol and various oils, including those of petroleum, animal, vegetable or synthetic origin, e.g., peanut oil, soybean oil, mineral oil, sesame oil, etc. Liquid carriers, particularly for injectable solutions, include water, saline, aqueous dextrose, and glycols. For examples of carriers, stabilizers, preservatives and adjuvants, see Remington's Pharmaceutical Sciences, edited by E. W. Martin (Mack Publishing Company, 18th ed., 1990). Additional excipients, for example sweetening, flavoring and coloring agents, may also be present.

The pharmaceutically acceptable excipient may be selected from the group consisting of fillers, e.g. sugars and/or sugar alcohols, e.g. lactose, sorbitol, mannitol, maltodextrin, etc.; surfactants, e.g. sodium lauryle sulfate, Brij 96 or Tween 80; disintegrants, e.g. sodium starch glycolate, maize starch or derivatives thereof; binder, e.g. povidone, crosspovidone, polyvinylalcohols, hydroxypropylmethylcellulose; lubricants, e.g. stearic acid or its salts; flowability enhancers, e.g. silicium dioxide; sweeteners, e.g. aspartame; and/or colorants. Pharmaceutically acceptable carriers include any and all clinically useful solvents, dispersion media, coatings, antibacterial and antifungal agents, isotonic and absorption delaying agents and the like.

The pharmaceutical composition may contain excipients for modifying, maintaining or preserving, for example, the pH, osmolarity, viscosity, clarity, color, isotonicity, odor, sterility, stability, rate of dissolution or release, adsorption or penetration of the composition. Suitable excipients include, but are not limited to, amino acids (such as glycine, glutamine, asparagine, arginine or lysine); antimicrobials; antioxidants (such as ascorbic acid, sodium sulfite or sodium hydrogen sulfite); buffers (such as borate, bicarbonate, Tris HCl, citrates, phosphates, other organic acids); bulking agents (such as mannitol or glycine), chelating agents (such as ethylenediamine tetraacetic acid (EDTA), ethylene glycol tetraacetic acid (EGTA)); complexing agents (such as caffeine, polyvinylpyrrolidone, beta cyclodextrin or hydroxypropyl beta cyclodextrin); fillers; monosaccharides; disaccharides and other carbohydrates (such as glucose, mannose, or dextrins); proteins (such as serum albumin, gelatin or immunoglobulins); coloring; flavoring and diluting agents; emulsifying agents; hydrophilic polymers (such as polyvinylpyrrolidone); low molecular weight polypeptides; salt forming counterions (such as sodium); preservatives (such as benzalkonium chloride, benzoic acid, salicylic acid, thimerosal, phenethyl alcohol, methylparaben, propylparaben, chlorhexidine, sorbic acid or hydrogen peroxide); solvents (such as glycerin, propylene glycol or polyethylene glycol); sugar alcohols (such as mannitol or sorbitol); suspending agents; surfactants or wetting agents (such as pluronics, PEG, sorbitan esters, polysorbates such as polysorbate 20, polysorbate 80, triton, tromethamine, lecithin, cholesterol, tyloxapal); stability enhancing agents (sucrose or sorbitol); tonicity enhancing agents (such as alkali metal halides (in one aspect, sodium or potassium chloride, mannitol sorbitol); delivery vehicles; diluents; excipients and/or pharmaceutical adjuvants. (Remington's Pharmaceutical Sciences, 18th Edition, A. R. Gennaro, ed., Mack Publishing Company, 1990).

Oral dosage forms may be tablets, capsules, bars, sachets, granules, syrups and aqueous or oily suspensions. Tablets may be formed form a mixture of the active compounds with fillers, for example calcium phosphate; disintegrating agents, for example maize starch, lubricating agents, for example magnesium stearate; binders, for example microcrystalline cellulose or polyvinylpyrrolidone and other optional ingredients known in the art to permit tabletting the mixture by known methods. Similarly, capsules, for example hard or soft gelatin capsules, containing the active compound, may be prepared by known methods. The contents of the capsule may be formulated using known methods so as to give sustained release of the active compounds. Other dosage forms for oral administration include, for example, aqueous suspensions containing the active compounds in an aqueous medium in the presence of a non-toxic suspending agent such as sodium carboxymethylcellulose, and oily suspensions containing the active compounds in a suitable vegetable oil, for example arachis oil. The active compounds may be formulated into granules with or without additional excipients. The granules may be ingested directly by the patient or they may be added to a suitable liquid carrier (e.g. water) before ingestion. The granules may contain disintegrants, e.g. an effervescent pair formed from an acid and a carbonate or bicarbonate salt to facilitate dispersion in the liquid medium. U.S. Pat. No. 8,263,662.

Intravenous forms include, but are not limited to, bolus and drip injections. Examples of intravenous dosage forms include, but are not limited to, Water for Injection USP; aqueous vehicles including, but not limited to, Sodium Chloride Injection, Ringer's Injection, Dextrose Injection, Dextrose and Sodium Chloride Injection, and Lactated Ringer's Injection; water-miscible vehicles including, but not limited to, ethyl alcohol, polyethylene glycol and polypropylene glycol; and non-aqueous vehicles including, but not limited to, corn oil, cottonseed oil, peanut oil, sesame oil, ethyl oleate, isopropyl myristate and benzyl benzoate.

Additional compositions include formulations in sustained or controlled delivery, such as using liposome or micelle carriers, bioerodible microparticles or porous beads and depot injections.

The present compound(s) or composition may be administered as a single dose, or as two or more doses (which may or may not contain the same amount of the desired molecule) over time, or as a continuous infusion via implantation device or catheter. The pharmaceutical composition can be prepared in single unit dosage forms.

Appropriate frequency of administration can be determined by one of skill in the art and can be administered once or several times per day (e.g., twice, three, four or five times daily). The compositions of the invention may also be administered once each day or once every other day. The compositions may also be given twice weekly, weekly, monthly, or semi-annually. In the case of acute administration, treatment is typically carried out for periods of hours or days, while chronic treatment can be carried out for weeks, months, or even years. U.S. Pat. No. 8,501,686.

Administration of the compositions of the invention can be carried out using any of several standard methods including, but not limited to, continuous infusion, bolus injection, intermittent infusion, inhalation, or combinations of these methods. For example, one mode of administration that can be used involves continuous intravenous infusion. The infusion of the compositions of the invention can, if desired, be preceded by a bolus injection.

The amount of the VHL inhibitor (e.g., a first amount) or the amount of the second agent (e.g., a second amount) that may be used in the combination therapy may be a therapeutically effective amount, a sub-therapeutically effective amount or a synergistically effective amount. The amounts are dosages that achieve the desired synergism.

As used herein, the term “therapeutically effective amount” is an amount sufficient to treat a specified disorder or disease or alternatively to obtain a pharmacological response treating a disorder or disease.

Methods of determining the most effective means and dosage of administration can vary with the composition used for therapy, the purpose of the therapy, the target cell being treated, and the subject being treated. Single or multiple administrations can be carried out with the dose level and pattern being selected by the treating physician. The specific dose level for any particular subject depends upon a variety of factors including the activity of the specific peptide, the age, body weight, general health, sex, diet, time of administration, route of administration, and rate of excretion, drug combination and the severity of the particular disease undergoing therapy.

For example, the present VHL inhibitor may be administered at about 0.0001 mg/kg to about 500 mg/kg, about 0.01 mg/kg to about 200 mg/kg, about 0.01 mg/kg to about 0.1 mg/kg, about 0.1 mg/kg to about 100 mg/kg, about 10 mg/kg to about 200 mg/kg, about 10 mg/kg to about 20 mg/kg, about 5 mg/kg to about 15 mg/kg, about 0.0001 mg/kg to about 0.001 mg/kg, about 0.001 mg/kg to about 0.01 mg/kg, about 0.01 mg/kg to about 0.1 mg/kg, about 0.1 mg/kg to about 0.5 mg/kg, about 0.5 mg/kg to about 1 mg/kg, about 1 mg/kg to about 2.5 mg/kg, about 2.5 mg/kg to about 10 mg/kg, about 10 mg/kg to about 50 mg/kg, about 50 mg/kg to about 100 mg/kg, about 100 mg/kg to about 250 mg/kg, about 0.1 μg/kg to about 800 μg/kg, about 0.5 μg/kg to about 500 μg/kg, about 1 μg/kg to about 20 μg/kg, about 1 μg/kg to about 10 ∞g/kg, about 10 μg/kg to about 20 μg/kg, about 20 μg/kg to about 40 μg/kg, about 40 μg/kg to about 60 μg/kg, about 60 μg/kg to about 100 μg/kg, about 100 μg/kg to about 200 μg/kg, about 200 μg/kg to about 300 μg/kg, or about 400 μg/kg to about 600 μg/kg. In some embodiments, the dose is within the range of about 250 mg/kg to about 500 mg/kg, about 0.5 mg/kg to about 50 mg/kg, or any other suitable amounts.

In certain embodiments, the amount or dose of the present VHL inhibitor may range from about 0.01 mg to about 10 g, from about 0.1 mg to about 9 g, from about 1 mg to about 8 g, from about 1 mg to about 7 g, from about 5 mg to about 6 g, from about 10 mg to about 5 g, from about 20 mg to about 1 g, from about 50 mg to about 800 mg, from about 100 mg to about 500 mg, from about 600 mg to about 800 mg, from about 800 mg to about 1 g, from about 0.01mg to about 10 g, from about 0.05 μg to about 1.5 mg, from about 10 μg to about 1 mg protein, from about 0.1 mg to about 10 mg, from about 2 mg to about 5 mg, from about 1 mg to about 20 mg, from about 30 μg to about 500 μg, from about 40 pg to about 300 pg, from about 0.1 μg to about 200 mg, from about 0.1 μg to about 5 μg, from about 5 μg to about 10 μg, from about 10 μg to about 25 μg, from about 25 μg to about 50 μg, from about 50 μg to about 100 μg, from about 100 μg to about 500 μg, from about 500 μg to about 1 mg, from about 1 mg to about 2 mg, e.g., in the pharmaceutical composition.

The dose of the present VHL inhibitor may range from about 0.1 μg/day to about 1 mg/day, from about 10 μg/day to about 200 μg/day, from about 20 μg/day to about 150 μg/day, from about 0.1 μg/day to about 125 μg/day, from about 1 μg/day to about 20 μg/day, or about 4.5 μg/day to about 30 μg/day.

Different dosage regimens may be used. In some embodiments, a daily dosage, such as any of the exemplary dosages described above, is administered once, twice, three times, or four times a day for at least three, four, five, six, seven, eight, nine, or ten days. Depending on the stage and severity of the cancer, a shorter treatment time (e.g., up to five days) may be employed along with a high dosage, or a longer treatment time (e.g., ten or more days, or weeks, or a month, or longer) may be employed along with a low dosage. In some embodiments, a once- or twice-daily dosage is administered every other day.

### Kits

In another aspect, the present invention provides any of the compositions described herein in kits, optionally including instructions for use of the compositions (e.g., for improving neuronal survival and/or inhibiting Vhl). That is, the kit can include a description of use of a composition in any method described herein. A “kit,” as used herein, typically defines a package, assembly, or container (such as an insulated container) including one or more of the components or embodiments of the invention, and/or other components associated with the invention, for example, as previously described. Each of the components of the kit may be provided in liquid form (e.g., in solution), or in solid form (e.g., a dried powder, frozen, etc.).

In some cases, the kit includes one or more components, which may be within the same or in two or more receptacles, and/or in any combination thereof. The receptacle is able to contain a liquid, and non-limiting examples include bottles, vials, jars, tubes, flasks, beakers, or the like. In some cases, the receptacle is spill-proof (when closed, liquid cannot exit the receptacle, regardless of orientation of the receptacle).

Examples of other compositions or components associated with the invention include, but are not limited to, diluents, salts, buffers, chelating agents, preservatives, drying agents, antimicrobials, needles, syringes, packaging materials, tubes, bottles, flasks, beakers, and the like, for example, for using, modifying, assembling, storing, packaging, preparing, mixing, diluting, and/or preserving the components for a particular use. In embodiments where liquid forms of any of the components are used, the liquid form may be concentrated or ready to use.

A kit of the invention generally will include instructions or instructions to a website or other source in any form that are provided for using the kit in connection with the components and/or methods of the invention. For instance, the instructions may include instructions for the use, modification, mixing, diluting, preserving, assembly, storage, packaging, and/or preparation of the components and/or other components associated with the kit. In some cases, the instructions may also include instructions for the delivery of the components, for example, for shipping at room temperature, sub-zero temperatures, cryogenic temperatures, etc. The instructions may be provided in any form that is useful to the user of the kit, such as written or oral (e.g., telephonic), digital, optical, visual (e.g., videotape, DVD, etc.) and/or electronic communications (including Internet or web-based communications), provided in any manner.

As used herein, instructions can include protocols, directions, guides, warnings, labels, notes, and/or “frequently asked questions” (FAQs), and typically involve written instructions on or associated with the invention and/or with the packaging of the invention. Instructions can also include instructional communications in any form (e.g., oral, electronic, digital, optical, visual, etc.), provided in any manner (e.g., within or separate from a kit) such that a user will clearly recognize that the instructions are to be used with the kit.

### Example 1

Experimental Design: Determine the effects of enhancing glycolytic metabolism in rods via gene therapy to assess the mechanism of attenuation in neurodegeneration.

We crossed a CreER RP mouse harboring mutations in Pde6b with a mouse floxed at Vhl to create an inducible gene therapy model. Upon tamoxifen injection, CreER is activated and excises Vhl exclusively in rod photoreceptors. We systematically injected tamoxifen in 7-, 8- and 10-day old mice to determine whether Vhl inhibition is neuroprotective as assessed by ERG, histology, immunostaining, and mass spectrometry.

### Results

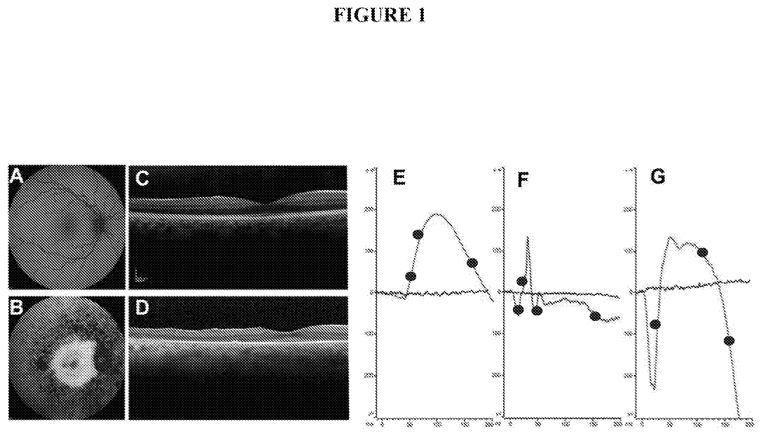

FIGS. 1A-1G provide a comparison of morphological and functional imaging modalities in control and RP patients. Autofluorescence imaging (FIGS. 1A-1B) and optical coherence tomography (FIGS. 1C-1D) from a control (top) and affected RP patient (bottom) are shown. Normal autofluorescence (FIG. 1A) as well as preserved retinal layer thickness (FIG. 1C) are observable in the control subject. In the affected RP patient, patchy hypo-autofluorescence in the periphery is indicative of RPE loss, with a characteristic foveal autofluorescent ring (FIG. 1B) as well as thinning of the outer nuclear layer (ONL) and the outer and inner segment layers (OS/IS) (FIG. 1D), indicating photoreceptor degeneration. In FIGS. 1E-1G, electroretinography (ERG) measures retinal function in response to light. Stimulation of the normal eye (black) elicits a well-formed scotopic (FIG. 1E), maximum (FIG. 1F), and photopic (FIG. 1G) response, while severe attenuation of the photodynamic response is evident in RP patients (curves with closed circles).

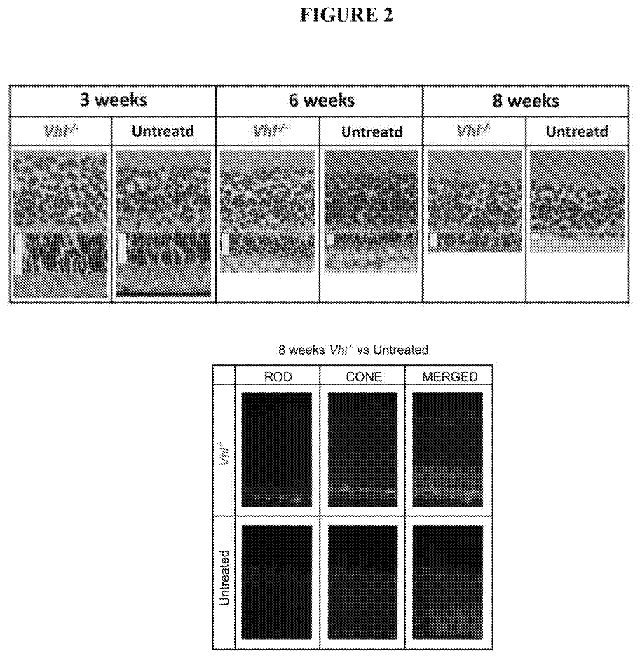

FIG. 2 shows that photoreceptor cells are preserved after genome surgery. The ONL (bar) was thicker in the Vhl^{-/-} group compared to the untreated group at 4, 6, and 8 weeks. The untreated group continued to experience progressive degeneration of the ONL, while dysgenesis was slowed in the Vhl^{-/-} mice. Immunostaining at 8 weeks showed a more robust cone (cone arrestin) and rod (rhodopsin) signal in the Vhl^{-/-} group compared to the weak cone and undetectable rod signal in untreated mice (dashed line indicates location of outer plexiform layer).

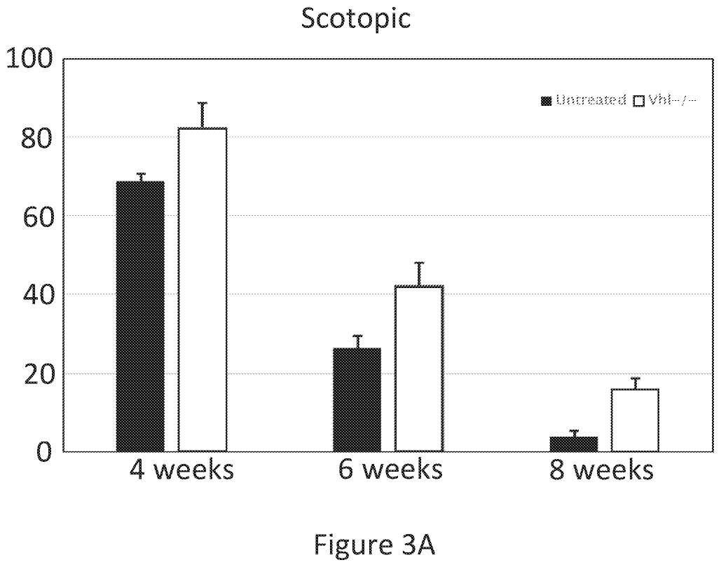

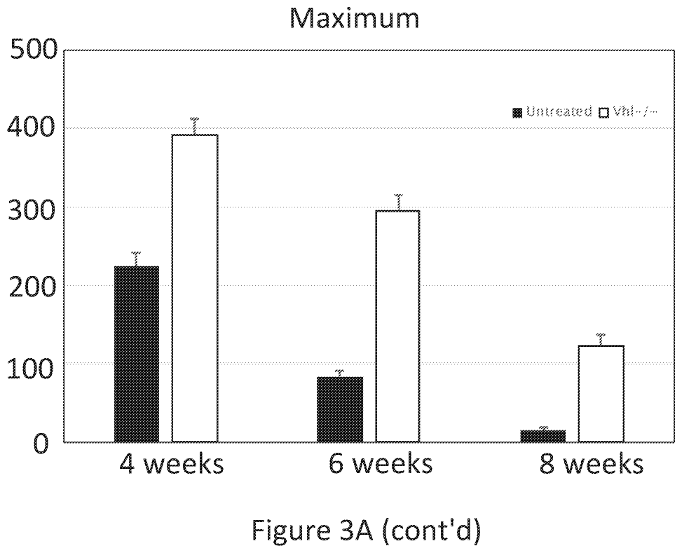

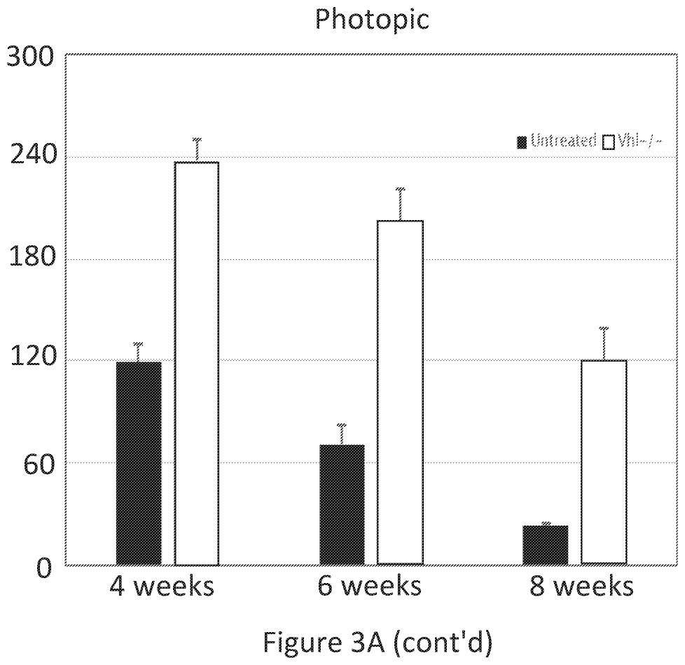

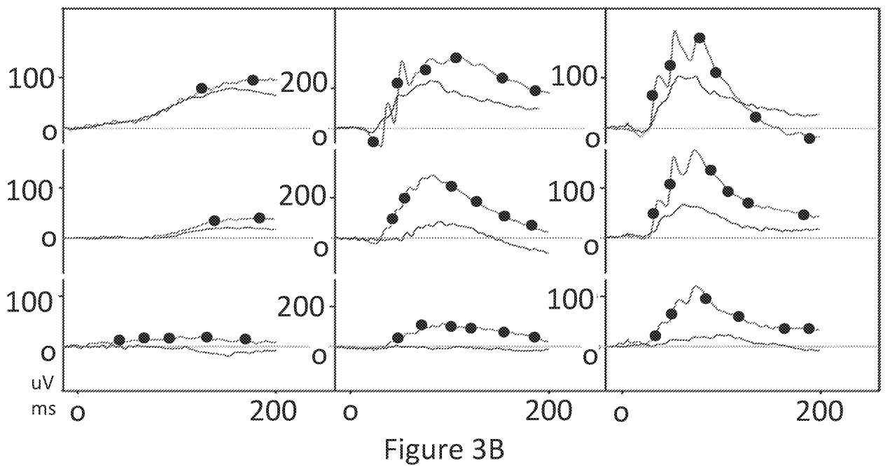

FIGS. 3A-B are graphs showing that photoreceptor function is preserved after genome surgery, as assessed by ERG. ERG in 4-, 6-, and 8-week old mice revealed higher scotopic, maximum, and photopic b-wave amplitudes (FIG. 3A) in the Vhl^{-/-} mice (white bar) compared with untreated mice (black), indicating that the function of the rods and cones is preserved in the treated group. Example raw data traces are also shown (FIG. 3B).

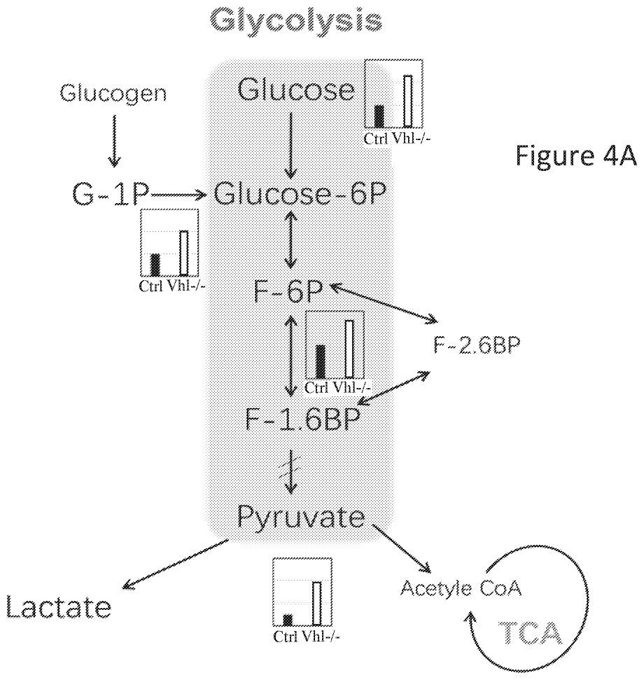

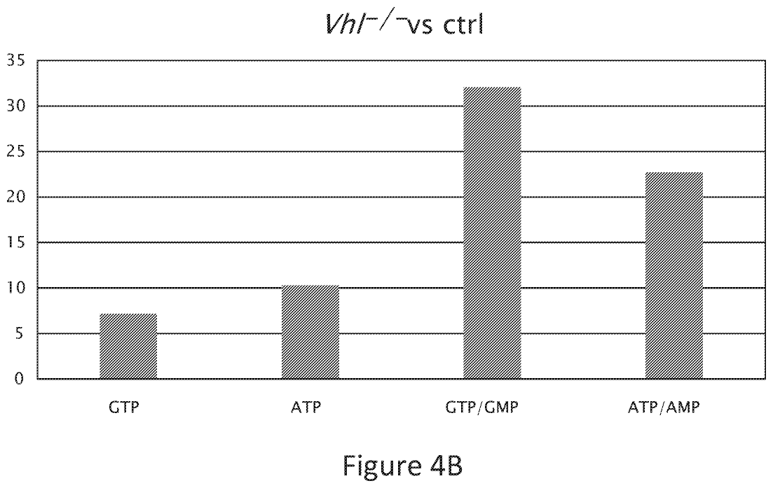

FIGS. 4A and 4B show that glycolysis is upregulated after genome surgery, resulting in higher levels of ATP. Mass spectrometry at 4 weeks revealed that the concentration of glucose, pyruvate, and FBP/GBP is higher in the Vhl^{-/-} group compared to the untreated mice, suggesting that glycolysis is upregulated after gene therapy. High-energy molecules like ATP and GTP were found in greater abundance in the Vhl^{-/-} group compared to the untreated mice.

Conclusions: Metabolic reprogramming through gene therapy increases glycolytic metabolism, leading to structural and functional perseveration of the retina in a preclinical model of RP.

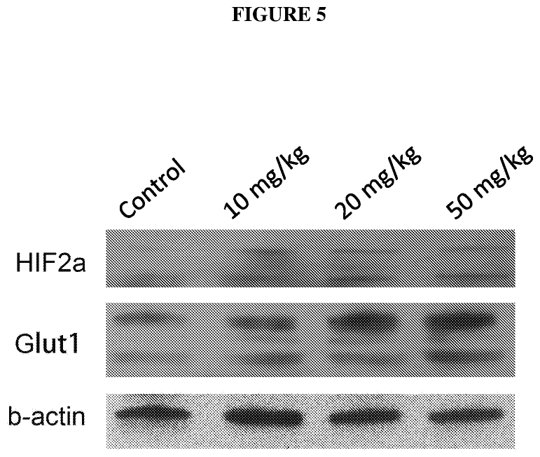

6-week old B6 mice were orally fed for 3 days with VH298 in oil (10, 20, or 50 mg/kg) or oil only for the control group. Each group contained 3 mice. 4 hours after the last feeding, mice retinas were collected and processed immediately for immunoblot. Protein levels of HIF2a, and Glut1 were shown in FIG. 5. The levels of HIF2a and GLUT1 showed a VH298 dose-dependent increase in mice treated with VH298, an inhibitor of VHL.

### Materials and Methods

### Animals

The Columbia University Institutional Animal Care and Use Committee (IACUC) approved all experiments prior to initiation. Mice were used in accordance with the Statement for the Use of Animals in Ophthalmic and Vision Research of the Association for Research in Vision and Ophthalmology and the Policy for the Use of Animals in Neuroscience Research of the Society for Neuroscience.

Three lines of mice were crossed to develop the breeding strains. Vhl^{tml.lCxd}/J mice (68) were purchased from the Jackson Laboratory; Ped6b^{H620Q}/Pde6b^{H620Q} mice were rederived via oviduct transfer using European Mouse Mutant Archive (EMMA) morulae (41, 69); and Pde6g^{CreERT2} mice were generated in the Barbara & Donald Jonas Stem Cell & Regenerative Medicine Laboratory (70-77). All mice were housed in the Columbia University Pathogen-free Eye Institute Annex Animal Care Services Facility and maintained with a 12-h light/12-h dark cycle.

Pde6b^{H620Q}/Pde6b^{H620Q} mice were crossed with Pde6g^{CreERT2} mice, and their offspring were bred with Vhl^{tm1.1Csd}/J mice. Six generations of backcrosses were required to generate breeding mice. The resulting progeny were homozygous for all alleles of interest (Pde6b, Vhl, and Pde6g), but some were wild type at Pde6g, whereas others possessed the Pde6g^{CreERT2} mutation. We isolated these two lines for use as breeding strains. Crossing the breeding strains produced the experimental mice, which are homozygous at the Pde6b and Vhl loci, and heterozygous at the Pde6g locus.

At P10, half of the experimental mice were given a 100 μg body weight (BW) injection of tamoxifen (100 mg/ml in ethanol; catalog T5648; Sigma-Aldrich), which was diluted with corn oil to a concentration of 10 mg/ml and thoroughly mixed at 42° C. One injection was administered on P10, 12, and 14. The other half of the experimental mice were injected with ethanol (10% in corn oil) following the same dosage as tamoxifen and served as the control group. There was no discrimination based on the sex of the mice.

### ONL Density and Inner/outer Segment Length Measurement

Mice were euthanized and eyes enucleated according to established IACUC guidelines following previously described procedures (72, 73). The cornea and lens were dissected and the vitreous removed, isolating the eyecup. Excalibur Pathology prepared H & E and retinal paraffin sections (5 μm). To quantify cell numbers and thickness, each section was divided into four regions: peripheral temporal; central temporal; central nasal; and peripheral nasal quadrants. ONL density was measured by counting the number of photoreceptor nuclei in each quadrant. This value was then divided by the length of the ONL measured in the four quadrants and the thickness of the section. The IS/OS length was determined by measuring the average thickness of the IS/OS layer in the four quadrants using Image J (NIH, Bethesda).

### Immunohistochemistry

For frozen sections, eyes were enucleated and placed in 4% paraformaldehyde for 1 h at room temperature. After fixation, retinae were dissected from the eyecup, cryoprotected in 30% glucose overnight at 4° C. and sectioned vertically at 10 μm with a cryostat (Leica). Sections were washed 3 times with phosphate-buffered saline (PBS, pH 7.4) and incubated overnight at 4° C. with the following primary antibodies: rabbit anti-cone arrestin (1:5000, Millipore), mouse anti-rhodopsin (1:500, Santa Cruz Biotechnology), rabbit anti-blue opsin (1:200, Millipore) and GFP-coupled anti-peanut agglutinin (PNA; 1:800, Millipore) diluted in 5% Chemiblocker (Life Technologies) and 0.3% Triton X-100 in PBS. After washing in PBS, the sections were incubated with secondary antibodies conjugated to either Alexa 555 or Alexa 488 (1:500, Molecular Probes, Life Technologies) for 1 h at room temperature. Sections were then washed with PBS and incubated for 5 min with 5 μg/ml Hoechst 33342 (Molecular Probes) and analyzed by confocal microscopy (Nikon A1). Only sections containing the optic nerve were included for analysis. Cone nuclei density was quantified manually on each section. Five sections through the optic nerve were collected and averaged for each mouse.

### ERG

After mice were dark-adapted overnight, recordings were obtained under dim red light illumination. Mice were anesthetized with an anesthetic solution (1 ml of 100 mg/ml ketamine and 0.1 ml of 20 mg/ml xylazine in 8.9 ml PBS) at a concentration of 0.1 ml/10 g BW injected in the intraperitoneal region. Heating pads were used to maintain body temperature at 37° C. One drop of Tropicamide Ophthalmic Solution (1%, Akorn) was administered in each eye for dilation. Ten minutes later, electrodes were placed on the corneas and Goniosol Hypromellose Ophthalmic Demulcent Solution (2.5%, Akron) was applied.

Both eyes were recorded simultaneously. Electrophysiological system (Diagnosys) was used to record ERG responses concurrently from both eyes. For rod and maximal rod and cone ERG responses, pulses of 0.00130 cd/m² and 3 cd/m² (White-6500K) were used. Each result represents the average of 40 to 60 trials. For cone responses, mice were light-adapted in the Ganzfeld dome for 10 min. A background of 30 cd/m² (White-6500K) was present throughout the trials to suppress rod function. ERGs were recorded using white flashes. ERGs were recorded at 4, 6, 8, and 10 weeks.

### ¹³C-Labeled Isotope Tracing and Mass Spectrometry

¹³C-labeled D-Glucose (U-¹³C₆, 99%) was purchased from Cambridge Isotope Laboratory, Inc. and was injected intraperitoneally (500 mg/Kg) in P21 control and experimental mice. After 45 minutes, retinae were promptly isolated from the mice, rinsed in PBS, and flash-frozen in liquid nitrogen. The retinae were subsequently homogenized in a mixture of methanol, chloroform, and water (700:200:50). The metabolites were dried, derivatized, and analyzed by GC-MS (Agilent 7890/5975C) as previously reported (78, 79). The chromatograms were analyzed using Agilent Chemstation software. The measured distribution of mass isotopologues was corrected for based on the natural abundance of isotopes using the software IsoCor. The fractional abundance of labeled ions to total ion intensity was determined.

Steady state metabolites were measured by LC-MS as previously reported by Du et al. 2015 (78). Three-week old control and experimental mice were sacrificed and retinae were collected, rinsed in PBS, and flash frozen in liquid nitrogen. Retinae were harvested at each time point and metabolites extracted in cold 80% methanol and quantified by Agilent 1260 LC (Agilent Technologies, Santa Clara, CA)-AB Sciex QTrap 5500 mass spectrometer (AB Sciex, Toronto, ON, Canada) system.

### REFERENCES

1. Hamel C. Retinitis pigmentosa. Orphanet journal of rare diseases. 2006;1(40.
2. Hartong D T, Berson E L, and Dryja T P. Retinitis pigmentosa. Lancet. 2006;368(9549):1795-809.
3. Shen B, Zhang W, Zhang J, Zhou J, Wang J, Chen L, Wang L, Hodgkins A, Iyer V, Huang X, et al. Efficient genome modification by CRISPR-Cas9 nickase with minimal off-target effects. Nature methods. 2014;11(4):399-402.
4. Wu W H, Tsai Y T, Justus S, Lee T, Zhang L, Lin C S, Bassuk A G, Mahajan V B, and Tsang S H. CRISPR repair reveals causative mutation in a preclinical model of retinitis pigmentosa. Mol Ther. 2016.
5. Tsang S H, Burns M E, Calvert P D, Gouras P, Baylor D A, Goff S P, and Arshaysky V Y. Role for the target enzyme in deactivation of photoreceptor G protein in vivo. Science. 1998;282(5386):117-21.
6. Tsang S H, Gouras P, Yamashita C K, Kjeldbye H, Fisher J, Farber D B, and Goff S P. Retinal degeneration in mice lacking the gamma subunit of the rod cGMP phosphodiesterase. Science. 1996;272(5264):1026-9.
7. Wert K J, Davis R J, Sancho-Pelluz J, Nishina P M, and Tsang S H. Gene therapy provides long-term visual function in a pre-clinical model of retinitis pigmentosa. Human molecular genetics. 2013;22(3):558-67.
8. Aherne A, Kennan A, Kenna P F, McNally N, Lloyd D G, Alberts I L, Kiang A S, Humphries M M, Ayuso C, Engel P C, et al. On the molecular pathology of neurodegeneration in IMPDH1-based retinitis pigmentosa. Human molecular genetics. 2004;13(6):641-50.
9. Wert K J, Lin J H, and Tsang S H. General pathophysiology in retinal degeneration. Developments in ophthalmology. 2014;53(33-43.
10. Dryja T P, Rucinski D E, Chen S H, and Berson E L. Frequency of mutations in the gene encoding the alpha subunit of rod cGMP-phosphodiesterase in autosomal recessive retinitis pigmentosa. Investigative ophthalmology & visual science. 1999;40(8):1859-65.
11. Davis R J, Tosi J, Janisch K M, Kasanuki J M, Wang N K, Kong J, Tsui I, Cilluffo M, Woodruff M L, Fain G L, et al. Functional rescue of degenerating photoreceptors in mice homozygous for a hypomorphic cGMP phosphodiesterase 6 b allele (Pde6bH620Q). Investigative ophthalmology & visual science. 2008;49(11):5067-76.
12. Punzo C, Kornacker K, and Cepko C L. Stimulation of the insulin/mTOR pathway delays cone death in a mouse model of retinitis pigmentosa. Nature neuroscience. 2009;12(1):44-52.
13. Venkatesh A, Ma S, Le Y Z, Hall M N, Ruegg M A, and Punzo C. Activated mTORC1 promotes long-term cone survival in retinitis pigmentosa mice. The Journal of clinical investigation. 2015;125(4):1446-58.
14. Lehninger A L, Nelson D L, and Cox M M. Lehninger principles of biochemistry. New York: W. H. Freeman; 2013.
15. Lin M K, Tsai Y-TT, and Tsang S H. Emerging Treatments for Retinitis Pigmentosa: Genes and stem cells, as well as new electronic and medical therapies, are gaining ground. Retinal physician. 2015;12(52-70.
16. Okawa H, Sampath A P, Laughlin S B, and Fain G L. ATP consumption by mammalian rod photoreceptors in darkness and in light. Current biology: CB. 2008;18(24):1917-21.
17. Nakanishi S. Second-order neurones and receptor mechanisms in visual-and olfactory-information processing. Trends in neurosciences. 1995;18(8):359-64.
18. Linsenmeier R A. Effects of light and darkness on oxygen distribution and consumption in the cat retina. The Journal of general physiology. 1986;88(4):521-42.
19. Ahmed J, Braun R D, Dunn R, Jr., and Linsenmeier R A. Oxygen distribution in the macaque retina. Investigative ophthalmology & visual science. 1993;34(3):516-21.
20. Birol G, Wang S, Budzynski E, Wangsa-Wirawan N D, and Linsenmeier R A. Oxygen distribution and consumption in the macaque retina. American journal of physiology Heart and circulatory physiology. 2007;293(3):H1696-704.
21. Du J, Rountree A, Cleghorn W M, Contreras L, Lindsay K J, Sadilek M, Gu H, Djukovic D, Raftery D, Satrustegui J, et al. Phototransduction influences metabolic flux and nucleotide metabolism in mouse retina. The Journal of biological chemistry. 2015.
22. Fariss R N, Li ZY, and Milam A H. Abnormalities in rod photoreceptors, amacrine cells, and horizontal cells in human retinas with retinitis pigmentosa. American journal of ophthalmology. 2000;129(2):215-23.
23. Curcio C A, Sloan K R, Kalina R E, and Hendrickson A E. Human photoreceptor topography. The Journal of comparative neurology. 1990;292(4):497-523.
24. Maguire A M, Simonelli F, Pierce E A, Pugh E N, Jr., Mingozzi F, Bennicelli J, Banfi S, Marshall K A, Testa F, Surace E M, et al. Safety and efficacy of gene transfer for Leber's congenital amaurosis. The New England journal of medicine. 2008;358(21):2240-8.
25. Ashtari M, Zhang H, Cook P A, Cyckowski L L, Shindler K S, Marshall K A, Aravand P, Vossough A, Gee J C, Maguire A M, et al. Plasticity of the human visual system after retinal gene therapy in patients with Leber's congenital amaurosis. Science translational medicine. 2015;7(296):296ra110.
26. Bainbridge J W, Smith A J, Barker S S, Robbie S, Henderson R, Balaggan K, Viswanathan A, Holder G E, Stockman A, Tyler N, et al. Effect of gene therapy on visual function in Leber's congenital amaurosis. The New England journal of medicine. 2008;358(21):2231-9.
27. Cideciyan A V, Hauswirth W W, Aleman T S, Kaushal S, Schwartz S B, Boye S L, Windsor E A, Conlon T J, Sumaroka A, Pang J J, et al. Human RPE65 gene therapy for Leber congenital amaurosis: persistence of early visual improvements and safety at 1 year. Human gene therapy. 2009;20(9):999-1004.
28. Maguire A M, High K A, Auricchio A, Wright J F, Pierce E A, Testa F, Mingozzi F, Bennicelli J L, Ying G S, Rossi S, et al. Age-dependent effects of RPE65 gene therapy for Leber's congenital amaurosis: a phase 1 dose-escalation trial. Lancet. 2009;374(9701):1597-605.
29. Simonelli F, Maguire A M, Testa F, Pierce E A, Mingozzi F, Bennicelli J L, Rossi S, Marshall K, Banfi S, Surace E M, et al. Gene therapy for Leber's congenital amaurosis is safe and effective through 1.5 years after vector administration. Molecular therapy:the journal of the American Society of Gene Therapy. 2010;18(3):643-50.
30. Jacobson S G, Cideciyan A V, Ratnakaram R, Heon E, Schwartz S B, Roman A J, Peden M C, Aleman T S, Boye S L, Sumaroka A, et al. Gene therapy for Leber congenital amaurosis caused by RPE65 mutations: safety and efficacy in 15 children and adults followed up to 3 years. Archives of ophthalmology. 2012;130(1):9-24.
31. Bennett J, Ashtari M, Wellman J, Marshall K A, Cyckowski L L, Chung D C, McCague S, Pierce E A, Chen Y, Bennicelli J L, et al. AAV2 gene therapy readministration in three adults with congenital blindness. Science translational medicine. 2012;4(120):120ra15.
32. Ashtari M, Cyckowski L L, Monroe J F, Marshall K A, Chung D C, Auricchio A, Simonelli F, Leroy B P, Maguire A M, Shindler K S, et al. The human visual cortex responds to gene therapy-mediated recovery of retinal function. The Journal of clinical investigation. 2011;121(6):2160-8.
33. Testa F, Maguire A M, Rossi S, Pierce E A, Melillo P, Marshall K, Banfi S, Surace E M, Sun J, Acerra C, et al. Three-year follow-up after unilateral subretinal delivery of adeno-associated virus in patients with Leber congenital Amaurosis type 2. Ophthalmology. 2013;120(6):1283-91.
34. Amado D, Mingozzi F, Hui D, Bennicelli J L, Wei Z, Chen Y, Bote E, Grant R L, Golden J A, Narfstrom K, et al. Safety and efficacy of subretinal readministration of a viral vector in large animals to treat congenital blindness. Science translational medicine. 2010;2(21):21ra16.
35. Vandenberghe L H, Bell P, Maguire A M, Cearley C N, Xiao R, Calcedo R, Wang L, Castle M J, Maguire A C, Grant R, et al. Dosage thresholds for AAV2 and AAV8 photoreceptor gene therapy in monkey. Science translational medicine. 2011;3(88):88ra54.
36. Daiger S P, Bowne S J, and Sullivan L S. Perspective on genes and mutations causing retinitis pigmentosa. Archives of ophthalmology. 2007;125(2):151-8.
37. Lin M, Kim S H, Zhang L, Tsai Y, and Tsang S H. Rod metabolic demand drives progression in retinopathies. Taiwan Journal of Ophthalmology. 2015;5(3):105-8.
38. Yau K W. Phototransduction mechanism in retinal rods and cones. The Friedenwald Lecture. Investigative ophthalmology & visual science. 1994;35(1):9-32.
41. Zwaans B M, and Lombard D B. Interplay between sirtuins, MYC and hypoxia-inducible factor in cancer-associated metabolic reprogramming. Dis Model Mech. 2014;7(9):1023-32.
68. Mohand-Said S, Hicks D, Dreyfus H, and Sahel J A. Selective transplantation of rods delays cone loss in a retinitis pigmentosa model. Archives of ophthalmology. 2000;118(6):807-11.
69. Xiong W, MacColl Garfinkel A E, Li Y, Benowitz L I, and Cepko C L. NRF2 promotes neuronal survival in neurodegeneration and acute nerve damage. The Journal of clinical investigation. 2015;125(4):1433-45.
70. Cepko C, and Punzo C. Cell metabolism: Sugar for sight. Nature. 2015;522(7557):428-9.
71. Cideciyan A V, Jacobson S G, Beltran W A, Sumaroka A, Swider M, Iwabe S, Roman A J, Olivares M B, Schwartz S B, Komaromy A M, et al. Human retinal gene therapy for Leber congenital amaurosis shows advancing retinal degeneration despite enduring visual improvement. Proceedings of the National Academy of Sciences of the United States of America. 2013;110(6):E517-25.
72. Jacobson S G, Cideciyan A V, Roman A J, Sumaroka A, Schwartz S B, Heon E, and Hauswirth W W. Improvement and decline in vision with gene therapy in childhood blindness. The New England journal of medicine. 2015;372(20): 1920-6.
73. Wright A F. Long-term effects of retinal gene therapy in childhood blindness. The New England journal of medicine. 2015;372(20):1954-5.
74. Du J, Rountree A, Cleghorn W M, Contreras L, Lindsay K J, Sadilek M, Gu H, Djukovic D, Raftery D, Satrustegui J, et al. Phototransduction Influences Metabolic Flux and Nucleotide Metabolism in Mouse Retina. The Journal of biological chemistry. 2016;291(9):4698-710.
75. Demetrius L A, Magistretti P J, and Pellerin L. Alzheimer's disease: the amyloid hypothesis and the Inverse Warburg effect. Front Physiol. 2014;5(522.
76. Demetrius L A, and Simon D K. An inverse-Warburg effect and the origin of Alzheimer's disease. Biogerontology. 2012;13(6):583-94.
77. Mariani AP. Neuronal and synaptic organization of the outer plexiform layer of the pigeon retina. The American journal of anatomy. 1987;179(1):25-39.
78. Masland R H. The neuronal organization of the retina. Neuron. 2012;76(2):266-80.
79. Kim H S, Xiao C, Wang R H, Lahusen T, Xu X, Vassilopoulos A, Vazquez-Ortiz G, Jeong W I, Park O, Ki SH, et al. Hepatic-specific disruption of SIRT6 in mice results in fatty liver formation due to enhanced glycolysis and triglyceride synthesis. Cell metabolism. 2010;12(3):224-36.
80. Hart A W, McKie L, Morgan J E, Gautier P, West K, Jackson I J, and Cross S H. Genotype-phenotype correlation of mouse pde6b mutations. Investigative ophthalmology & visual science. 2005;46(9):3443-50.
81. Koch S F, Tsai Y T, Duong J K, Wu W H, Hsu C W, Wu W P, Bonet-Ponce L, Lin C S, and Tsang S H. Halting progressive neurodegeneration in advanced retinitis pigmentosa. The Journal of clinical investigation. 2015;125(9):3704-13.
82. Tsang S H, Chen J, Kjeldbye H, Li W S, Simon M I, Gouras P, and Goff S P. Retarding photoreceptor degeneration in Pdegtml/Pdegtml mice by an apoptosis suppressor gene. Investigative ophthalmology & visual science. 1997;38(5):943-50.
83. Tanabe T, Tsang S H, Kjeldbye H, Berns K, Goff S, and Gouras P. Adeno-associated virus mediated gene transfer into pde g knockout mouse. Investigative ophthalmology & visual science. 1998:S5153.
84. Tosi J, Davis R J, Wang N K, Naumann M, Lin C S, and Tsang S H. shRNA knockdown of guanylate cyclase 2e or cyclic nucleotide gated channel alpha 1 increases photoreceptor survival in a cGMP phosphodiesterase mouse model of retinitis pigmentosa. J Cell Mol Med. 2011;15(8):1778-87.
85. Tsang S H, Tsui I, Chou C L, Zernant J, Haamer E, Iranmanesh R, Tosi J, and Allikmets R. A novel mutation and phenotypes in phosphodiesterase 6 deficiency. American journal of ophthalmology. 2008;146(5):780-8.
86. Tsang S, Woodruff M, Lin C, Jacobson B, Naumann M, Hsu C, Davis R, Cilluffo M C, Chen J, and Fain G. Effect of the ILE86TER mutation in the γ subunit of cGMP phosphodiesterase (PDE6) on rod photoreceptor signalling. Cellular Signalling. 2012;0(0.
87. Du J, Linton J D, and Hurley J B. Probing Metabolism in the Intact Retina Using Stable Isotope Tracers. Methods in enzymology. 2015;561(149-70.
88. Du J, Cleghorn W, Contreras L, Linton J D, Chan G C, Chertov A O, Saheki T, Govindaraju V, Sadilek M, Satrustegui J, et al. Cytosolic reducing power preserves glutamate in retina. Proceedings of the National Academy of Sciences of the United States of America. 2013;110(46):18501-6.

Many modifications and variations of this invention can be made without departing from its spirit and scope, as will be apparent to those skilled in the art. The invention is defined by the terms of the appended claims, along with the full scope of equivalents to which such claims are entitled. The specific embodiments described herein, including the following examples, are offered by way of example only, and do not by their details limit the scope of the invention.

All references cited herein are incorporated by reference to the same extent as if each individual publication, database entry (e.g. Genbank sequences or GenelD entries), patent application, or patent, was specifically and individually indicated to be incorporated by reference. This statement of incorporation by reference is intended by Applicants, pursuant to 37 C.F.R. § 1.57(b)(1), to relate to each and every individual publication, database entry (e.g. Genbank sequences or GenelD entries), patent application, or patent, each of which is clearly identified in compliance with 37 C.F.R. § 1.57(b)(2), even if such citation is not immediately adjacent to a dedicated statement of incorporation by reference. The inclusion of dedicated statements of incorporation by reference, if any, within the specification does not in any way weaken this general statement of incorporation by reference. Citation of the references herein is not intended as an admission that the reference is pertinent prior art, nor does it constitute any admission as to the contents or date of these publications or documents.

The present invention is not to be limited in scope by the specific embodiments described herein. Indeed, various modifications of the invention in addition to those described herein will become apparent to those skilled in the art from the foregoing description and the accompanying figures. Such modifications are intended to fall within the scope of the appended claims. The foregoing written specification is considered to be sufficient to enable one skilled in the art to practice the invention. Various modifications of the invention in addition to those shown and described herein will become apparent to those skilled in the art from the foregoing description and fall within the scope of the appended claims.

1. A method of increasing glycolysis in a neuronal cell, the method comprising inhibiting or decreasing level and/or activity of VHL in the neuronal cell.

2. The method of claim 1, wherein the neuronal cell is a cone cell or a rod cell, or a combination of cone cells, rod cells, and/or other retinal cells.

3. The method of claim 1, wherein the inhibiting or decreasing comprises administering an effective amount of an inhibitor of VHL.

4. The method of claim 1, wherein the inhibitor of VHL is selected from the group consisting of proteins, nucleic acids, chemicals and combinations thereof.

5. The method of claim 4, wherein the nucleic acid is selected from the group consisting of antisense oligonucleotide, a small interfering RNA (siRNA), a short hairpin RNA (shRNA), a guide RNA (gRNA) and combinations thereof.

6. The method of claim 3, wherein the inhibitor of VHL is VH298 ((2S,4R)-1-((S)-2-(1-cyanocyclopropanecarboxamido)-3,3-dimethylbutanoyl)-4-hydroxy-N-(4-(4-methylthiazol-5-yl)benzyl)pyrrolidine-2-carboxamide), and/or VH032 ((2S,4R)-1-((S)-2-acetamido-3,3-dimethylbutanoyl)-4-hydroxy-N-(4-(4-methylthiazol-5-yl)benzyl)pyrrolidine-2-carboxamide).

7. The method of claim 1, wherein the inhibiting or decreasing comprises administering an effective amount of any combination of inhibitors of VHL.

8. A method of increasing neuronal survival and/or photoreceptor survival in a patient in need thereof, the method comprising administering an effective amount of an inhibitor of VHL to the patient.

9-10. (canceled)

11. The method of claim 8, wherein the inhibitor of VHL is selected from the group consisting of proteins, nucleic acids, chemicals and combinations thereof.

12. The method of claim 11, wherein the nucleic acid is selected from the group consisting of antisense oligonucleotide, a small interfering RNA (siRNA), a short hairpin RNA (shRNA), a guide RNA (gRNA) and combinations thereof.

13. The method of claim 8, wherein the inhibitor of VHL is VH298 ((2S,4R)-1-((S)-2-(1-cyanocyclopropanecarboxamido)-3,3-dimethylbutanoyl)-4-hydroxy-N-(4-(4-methylthiazol-5-yl)benzyl)pyrrolidine-2-carboxamide), and/or VH032 ((2S,4R)-1-((S)-2-acetamido-3,3-dimethylbutanoyl)-4-hydroxy-N-(4-(4-methylthiazol-5-yl)benzyl)pyrrolidine-2-carboxamide).

14. (canceled)

15. The method of claim 8, wherein the patient is suffering from at least one retinal degenerative disease.

16. The method of claim 15, wherein the retinal degenerative disease is retinitis pigmentosa (RP), age-related macular degeneration (AMD), and/or glaucoma.

17. The method of claim 8, wherein the patient is suffering from at least one neurodegenerative disease.

18. The method of claim 17, wherein the neurodegenerative disease is Alzheimer's disease, Parkinson's disease, Huntington's disease, Amyotrophic lateral sclerosis (ALS), and/or Lewy body dementia.

19-24. (canceled)

25. The method of claim 8, wherein the inhibitor of VHL is encoded by a recombinant adeno-associated viral (AAV) vector.

26. The method of claim 25, wherein the recombinant AAV vector is an AAV2 vector or an AAV8 vector.

27. (canceled)

28. The method of claim 25, wherein the AAV vectors are administered by intravitreal injection or subretinal injection.

29. (canceled)

30. The method of claim 8, wherein the inhibitor of VHL is at least one guide RNA that hybridizes to the endogenous Vhl gene in the patient, wherein the at least one guide RNA is encoded by a first recombinant adeno-associated viral (AAV) vector, and wherein the method further comprises administering to the patient a second recombinant AAV viral vector comprising a nucleic acid sequence encoding a Cas nuclease; wherein the Cas nuclease cleaves the endogenous Vhl gene creating a Vhl knockout of the endogenous Vhl gene in the patient.

31-32. (canceled)

33. The method of claim 30, wherein the Cas nuclease is Cas9.

34-57. (canceled)
